(19) United States
(12) Patent Application Publication
(10) Pub. No.: US 2025/0049717 A1
(43) Pub. Date: Feb. 13, 2025

(54) DRY POWDER FORMULATIONS OF NARROW SPECTRUM KINASE INHIBITORS

(71) Applicant: Pulmatrix Operating Company, Inc., Bedford, MA (US)

(72) Inventors: Jason M. PERRY, Somerville, MA (US); Aidan K. CURRAN, Duxbury, MA (US); Hillary T. BERGSON, Wilmington, MA (US)

(21) Appl. No.: 18/719,947

(22) PCT Filed: Dec. 19, 2022
(86) PCT No.: PCT/US2022/081909

§ 371 Date: Jun. 14, 2024

Related U.S. Application Data
(60) Provisional application No. 63/291,479, filed on Dec. 20, 2021.
(60) Provisional application No. 63/321,418, filed on Mar. 18, 2022.

(51) Int. Cl. A61K 9/16; A61K 9/00; A61K 31/506

(52) U.S. Cl. CPC A61K 9/1623; A61K 9/0075; A61K 9/1611; A61K 31/506

(57) ABSTRACT

The present disclosure relates to respirable dry powders comprising respirable dry particles that comprise, a stabilizer and one or more excipients.

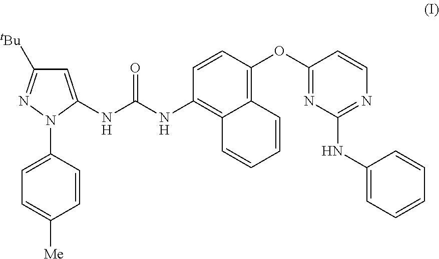 [SMILES: Cc1ccc(-n2nc(C(C)(C)C)cc2NC(=O)Nc2ccc(Oc3ccnc(Nc4ccccc4)n3)c3ccccc23)cc1]

### RELATED APPLICATIONS

This application claims the benefit of U.S. Provisional Application No. 63/291,479, filed on Dec. 20, 2021, and U.S. Provisional Application No. 63/321,418, filed on Mar. 18, 2022, the contents of which are each incorporated herein by reference in their entireties.

### BACKGROUND

The p38 mitogen-activated protein kinases (referred to herein as p38 MAP kinase) as well as Src and Syk family of tyrosine kinases are known to play a role in inflammation. There are several isoforms of p38 MAP kinase with broad tissue distribution, and early classes of p38 MAPK inhibitors were highly toxic. A major obstacle hindering the utility of p38 MAP kinase inhibitors in the treatment of human diseases has been the severe toxicity observed in patients. See. e.g., Pettus, L. H. and Wurz, R. P., Curr. Top. Med. Chem., 2008, 8(16):1452-67).

p38 MAP kinase is believed to play a pivotal role in many of the signaling pathways that are involved in initiating and maintaining human disease. For example, p38 MAP kinase is thought be involved in chronic, persistent inflammation in severe asthma and in chronic obstructive pulmonary disease (COPD) (Chung, F., Chest, 2011, 139(6):1470-1479). Members of the Src family (Hck, Fgr and Lyn) has been reported to accelerate clearance of Pneumocystis murina infection from the lungs of mice. See. e.g., Nelson, M. P., et al., Infection and Immunity, 2009, 77(5): p. 1790-1797. In addition, Fyn, one of the Src family of kinases is also reported to be involved in replication of Dengue virus, which is an RNA virus similar to human rhinovirus (HRV). De Wispelaere, M., A. J. Lacroix, and P. L. Yang, Journal of Virology, 2013, 87(13): p. 7367-7381. Consistent with the findings for RV 1162 described here, Syk kinase has been reported to be activated in epithelial cells exposed to HRV. Wang, X., et al., J Pharmacol Exp Ther, 2012, 340(2): p. 277-85; Lau, C., et al., Protein Pept Lett, 2011, 18(5): p. 518-29.

An experimental Narrow Spectrum Kinase Inhibitor (NSKI) formulation for inhalation was previously developed, which included an inhibitor of p38 MAP, Src and Syk kinases blended with lactose. However, that lactose blend formulation did not progress beyond phase 1 clinical trials (see NCT01970618). As shown in studies described herein, administering the lactose blend to the respiratory tract of a subject results in a high lung:plasma ratio, followed by significant accumulation with repeat doses, which is believed to be due to slow dissolution in the lungs. Consequently, the inhibition of kinase activity in the lung, as demonstrated by levels of p38 MAP kinase phosphorylation, occurs secondary to the significant accumulation with repeat doses. With chronic dosing of the lactose blend there is initially a low systemic level of the NSKI, and high levels in the lungs, which leads to a prolonged post-dose exposure. Ultimately because of these poor pharmacokinetics, the lactose blend formulation has insufficient safety margin and is unsuitable for chronic dosing.

A need exists for new formulations of kinase inhibitors that exhibit improved kinetics, have no or low toxicity at the relevant therapeutic dose, and can safely be administered to subjects, e.g., to treat diseases or disorders associated with the aforementioned kinases, such as inflammations, including with chronic dosing.

The present disclosure provides a method for treatment of respiratory disorders such as asthma and chronic obstructive pulmonary disease. Embodiments of the present disclosure provide a dry powder pharmaceutical formulation comprising a nanocrystalline active ingredient that has been optimized to maximize dissolution rate and enhanced absorption into the lung tissues.

### SUMMARY

The present disclosure relates to respirable dry powders comprising (e.g., consisting of) homogenous respirable dry particles that comprise (e.g., consist of) an active ingredient, a stabilizer, and one or more excipients, wherein the active ingredient is Compound A, the active ingredient is present in an amount of about 0.5% to about 20% by weight, the ratio of the stabilizer to the active ingredient is about 1:5 to about 1:20 (wt %:wt %) and the one or more excipients comprise mannitol and sodium sulfate in an amount of about 4:1 to about 1:4 (wt %:wt %). Optionally, other or additional excipients are used.

The present disclosure also relates to respirable dry powders comprising (e.g., consisting of) homogenous respirable dry particles that comprise (e.g., consist of) an active ingredient and one or more excipients, wherein the active ingredient is Compound A, the active ingredient is present in an amount of about 0.5% to about 20% by weight, the ratio of the stabilizer to the active ingredient is about 1:5 to about 1:20 (wt %:wt %) and the one or more excipients comprise mannitol and sodium sulfate in an amount of about 4:1 to about 1:4 (wt %:wt %). Optionally, other or additional excipients are used.

The respirable dry powders of the present disclosure are well tolerated following administration to the respiratory tract of a subject, including after repeat administrations (e.g., chronic dosing). The respirable dry powders of the present disclosure are rapidly dissolved in the lungs, which results in high initial systemic exposure without significant accumulation, providing a more constant systemic exposure over time with repeat dosing. As such, the respirable dry powders of the present disclosure have a high therapeutic index and can be safely administered in lower nominal doses relative to the lactose blend, and are suitable for chronic dosing.

The active ingredient is Compound A of Formula (I):

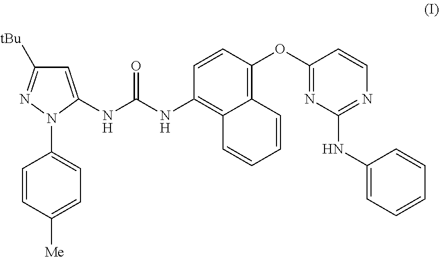 [SMILES: Cc1ccc(-n2nc(C(C)(C)C)cc2NC(=O)Nc2ccc(Oc3ccnc(Nc4ccccc4)n3)c3ccccc23)cc1]

or a pharmaceutically acceptable salt thereof, including all stereoisomers and tautomers thereof.

In some embodiments the Compound A of Formula (I) is in a crystalline particulate form.

In some embodiments the crystalline particulate form is a nano-crystalline form.

In some embodiments the nano-crystalline form is a sub-particle that comprises crystalline Compound A and optionally a stabilizer.

The sub-particle may be about 50 nm to about 500 nm (Dv50), about 50 nm to about 100 nm (Dv50), about 100 nm to about 200 nm (Dv50), or about 100 nm to about 150 nm (Dv50).

The active ingredient may be present in an amount of about 1% to about 20% by weight, about 2% to about 12% by weight, or about 2% to about 10% by weight.

In some embodiments the stabilizer is polysorbate 80.

The ratio of the stabilizer to the active ingredient (wt %:wt %) may be from about 1:5 to 1:20, about 1:5 to about 1:15, about 1:15 to about 1:20, or about 1:10.

The polysorbate 80 may be present in an amount less than 5% by weight, about 0.2% to about 2% by weight, about 0.2% to less than 5% by weight, or about 0.1% to less than 5% by weight.

The dry powder may further comprise one or more alternative or additional excipients selected from the group consisting of a monovalent metal cation salt, a divalent metal cation salt, an amino acid, a sugar alcohol, or combinations thereof.

In some embodiments the one or more alternative or additional excipients comprise a sodium salt and optionally at least one of an amino acid, a carbohydrate, a sugar alcohol, or combinations thereof.

In some embodiments the sodium salt is selected from the group consisting of sodium chloride, sodium citrate, and sodium sulfate, and the amino acid is leucine. The sodium salt may be sodium chloride and the amino acid may be leucine. The sodium salt may be sodium sulfate and the amino acid may be leucine.

In some embodiments, the one or more alternative or additional excipients are mannitol and sodium sulfate (e.g., in equal amounts).

The one or more alternative or additional excipients may comprise a magnesium salt and an amino acid. The magnesium salt may be magnesium lactate, and the amino acid may be leucine.

The present disclosure further relates to a dry powder pharmaceutical formulation comprising (e.g., consisting of) homogenous respirable dry particles that comprise (e.g., consist of): a) Compound A in a nanocrystalline form (2%), polysorbate 80 (0.2%), mannitol (48.9%) and sodium sulfate (48.9%), where all percentages are by weight of the dry particles (wt %); b) Compound A in a nanocrystalline form (5%), polysorbate 80 (0.5%), mannitol (47.25%) and sodium sulfate (47.25%), where all percentages are by weight of the dry particles (wt %); c) Compound A in a nanocrystalline form (10%), polysorbate 80 (1.0%), mannitol (44.5%) and sodium sulfate (44.5%), where all percentages are by weight of the dry particles (wt %); d) Compound A in a nanocrystalline form (7.6%), polysorbate 80 (0.8%), mannitol (44.5%) and sodium sulfate (44.5%), where all percentages are by weight of the dry particles (wt %); or e) Compound A in a nanocrystalline form (8.0%), polysorbate 80 (1.0%), mannitol (44.5%) and sodium sulfate (44.5%), where all percentages are by weight of the dry particles (wt %).

The present disclosure further relates to a dry powder pharmaceutical formulation comprising (e.g., consisting of) homogenous respirable dry particles that comprise (e.g., consist of): Compound A in a nanocrystalline form (5%), polysorbate 80 (0.5%), mannitol (47.25%) and sodium sulfate (47.25%), where all percentages are by weight of the dry particles (wt %).

The present disclosure further relates to a dry powder pharmaceutical formulation comprising (e.g., consisting of) homogenous respirable dry particles that comprise (e.g., consist of): Compound A in a nanocrystalline form (10%), polysorbate 80 (1.0%), mannitol (44.5%) and sodium sulfate (44.5%), where all percentages are by weight of the dry particles (wt %).

The respirable dry particles may have a volume median geometric diameter (VMGD) of about 10 microns or less, or about 5 microns or less. For example, the respirable dry particles may have a VMGD of about 1 micron, about 2 microns, about 3 microns, about 4 microns, or about 5 microns.

The respirable dry particles may have a tap density of about 0.2 g/cc or greater, a tap density of between 0.2 g/cc and 1.0 g/cc, or a tap density of greater than about 0.4 g/cc to about 1.2 g/cc. For example, the respirable dry particles may have a tap density of about 0.2 g/cc, about 0.3 g/cc, about 0.4 g/cc, about 0.5 g/cc, about 0.6 g/cc, about 0.7 g/cc, about 0.8 g/cc, about 0.9 g/cc, about 1.0 g/cc, about 1.1 g/cc, or about 1.2 g/cc.

The dry powder may have an MMAD of between about 1 micron and about 5 microns. For example, the dry powder may have an MMAD of about 1 micron, about 2 microns, about 3 microns, about 4 microns, or about 5 microns.

The dry particles may have a 1 bar/4 bar dispersibility ratio (1/4 bar) of less than about 1.5 as measured by laser diffraction. For example, the dry particles may have a 1 bar/4 bar dispersibility ratio (1/4 bar) of about 1.0, about 1.1, about 1.2, about 1.3, about 1.4, or about 1.5, as measured by laser diffraction.

The dry particles may have a 0.5 bar/4 bar dispersibility ratio (0.5/4 bar) of about 1.5 or less as measured by laser diffraction. For example, the dry particles may have a 0.5 bar/4 bar dispersibility ratio (0.5/4 bar) of about 1.0, about 1.1, about 1.2, about 1.3, about 1.4, or about 1.5, as measured by laser diffraction.

The dry powder may have a FPF of the total dose less than 5 microns of about 25% or more. For example, the dry powder may have a FPF of the total dose less than 5 microns of about 25%, about 50%, about 60%, about 70%, about 80%, or more.

The dry powder may be delivered to a subject (e.g., a patient) with a capsule-based passive dry powder inhaler.

In one aspect the present disclosure relates to a method for treatment or prevention of exacerbations in subjects (e.g., patients) with chronic respiratory disease, such as COPD (including chronic bronchitis and emphysema), asthma, pediatric asthma, cystic fibrosis, sarcoidosis, idiopathic pulmonary fibrosis comprising administering to a subject (e.g., a patient) in need thereof an effective amount of a dry powder or a dry powder pharmaceutical formulation described herein. The COPD may be stable COPD.

In one aspect the present disclosure relates to a method for treatment or prevention of a condition selected from: COPD (including chronic bronchitis and emphysema), asthma, pediatric asthma, cystic fibrosis, sarcoidosis, idiopathic pulmonary fibrosis, allergic rhinitis, rhinitis, sinusitis, allergic conjunctivitis, conjunctivitis, allergic dermatitis, contact dermatitis, psoriasis, ulcerative colitis, inflamed joints secondary to rheumatoid arthritis or osteoarthritis, rheumatoid arthritis, pancreatitis, cachexia, lung cancer, inhibition of the growth and metastasis of tumors including non-small cell lung carcinoma, breast carcinoma, gastric carcinoma, colorectal carcinomas and malignant melanoma comprising administering to a subject (e.g., a patient) in need thereof an effective amount of a dry powder or a dry powder pharmaceutical formulation described herein. The COPD may be stable COPD.

In one aspect, the present disclosure relates to a method for treatment or prevention of respiratory viral infections in subjects (e.g., patients) with chronic conditions such as congestive heart failure, diabetes, cancer, or in immunosuppressed subjects (e.g., patients), for example post-organ transplant comprising administering to a subject (e.g., a patient) in need thereof an effective amount of a dry powder or a dry powder pharmaceutical formulation described herein.

In one aspect, the present disclosure relates to a method for treatment or prevention of a condition selected from exacerbation of COPD and exacerbation of asthma comprising administering to a subject (e.g., a patient) in need thereof an effective amount of a dry powder or a dry powder pharmaceutical formulation described herein. The COPD may be stable COPD.

In one aspect, the exacerbation of COPD or exacerbation of asthma is a virally induced exacerbation.

In one aspect, the present disclosure relates to a method for treatment of exacerbation of inflammatory disease in subjects (e.g., patients) with chronic conditions such as selected from a group consisting of congestive heart failure, diabetes, cancer and conditions suffered by immunosuppressed subjects (e.g., patients) comprising administering to a subject (e.g., a patient) in need thereof an effective amount of a dry powder or a dry powder pharmaceutical formulation described herein.

In one aspect, the present disclosure relates to a method for treatment or prevention of acute exacerbation of a respiratory disease comprising administering to a subject (e.g., a patient) in need thereof an effective amount of a dry powder or a dry powder pharmaceutical formulation described herein.

In one aspect, the present disclosure relates to a dry powder or a dry powder pharmaceutical formulation produced by spray drying.

In another aspect, the present disclosure relates to a dry powder or a dry powder pharmaceutical formulation produced by a process comprising the steps of: spray drying a surfactant-stabilized suspension with optional excipients, wherein dry particles that are compositionally homogeneous are produced.

In another aspect, the present disclosure relates to a method for treating or preventing exacerbations in subjects (e.g., patients) with a chronic respiratory disease, such as COPD (including chronic bronchitis and emphysema), e.g., stable COPD, comprising administering to a subject (e.g., a patient) in need thereof an effective amount of a dry powder that comprises (e.g., consists of) homogenous respirable dry particles that comprise (e.g., consist of): (a) Compound A in a nanocrystalline form (5 wt %), polysorbate 80 (0.5 wt %), mannitol (47.25 wt %) and sodium sulfate (47.25 wt %); or (b) Compound A in a nanocrystalline form (10 wt %), polysorbate 80 (1.0 wt %), mannitol (44.5 wt %) and sodium sulfate (44.5 wt %).

In another aspect, the present disclosure relates to a method for treating or preventing COPD (e.g., chronic bronchitis and emphysema, or stable COPD), comprising administering to a subject (e.g., a patient) in need thereof an effective amount of a dry powder that comprises (e.g., consists of) homogenous respirable dry particles that comprise (e.g., consist of): (a) Compound A in a nanocrystalline form (5 wt %), polysorbate 80 (0.5 wt %), mannitol (47.25 wt %) and sodium sulfate (47.25 wt %); or (b) Compound A in a nanocrystalline form (10 wt %), polysorbate 80 (1.0 wt %), mannitol (44.5 wt %) and sodium sulfate (44.5 wt %).

In some embodiments, a dry powder described herein does not comprise lactose. In some embodiments, a dry particle described herein does not comprise lactose.

### BRIEF DESCRIPTION OF THE DRAWINGS

FIG. 1 shows an X-ray powder diffraction (XRPD) pattern obtained from Formulations VII and VIII.

FIG. 2 shows an XRPD pattern obtained from Formulations IX, X, and XI prepared in Example 3.

FIG. 3 shows an XRPD pattern obtained from Formulation XII.

FIG. 4 shows an XRPD pattern obtained from Formulation X and Formulation XI prepared in Example 10.

### DETAILED DESCRIPTION

This disclosure relates to respirable dry powders that contain 1-(3-tert-butyl-1-p-tolyl-1H-pyrazol-5-yl)-3-(4-(2-(phenylamino)pyrimidin-4-yloxy)naphthalen-1-yl)urea, including pharmaceutically acceptable salts and all enantiomers, tautomers and prodrugs thereof, as a pharmaceutically active ingredient. The compound 1-(3-tert-butyl-1-p-tolyl-1H-pyrazol-5-yl)-3-(4-(2-(phenylamino)pyrimidin-4-yloxy)naphthalen-1-yl)urea (referred to herein as “Compound A”) is a p38 MAP kinase inhibitor and is disclosed in WO 2013/050757 A1 and U.S. Pat. Nos. 9,850,231, 9,108,950, 10,266,519, and 10,738,032, each of these documents are incorporated herein by reference.

Compound A has the structure:

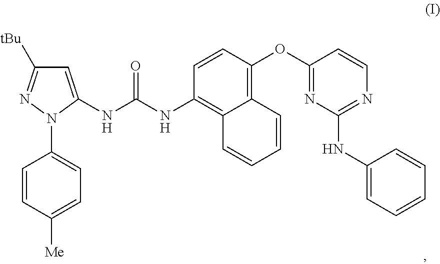 [SMILES: Cc1ccc(-n2nc(C(C)(C)C)cc2NC(=O)Nc2ccc(Oc3ccnc(Nc4ccccc4)n3)c3ccccc23)cc1]

where “tBu” is tert-butyl, and “Me” is methyl.

As disclosed in WO 2013/050757 A1, Compound A is a narrow spectrum kinase inhibitor that inhibits p38 MAP kinases enzymes, for example the alpha and gamma kinase sub-types thereof, and of Syk kinase and the Src family of tyrosine kinases. Examples of salts of Compound A include all pharmaceutically acceptable salts, such as, without limitation, acid addition salts of strong mineral acids such as hydrochloric acid (HCl) and hydrobromic acid (HBr), salts, and addition salts of strong organic acids such as methanesulfonic acid, or a pharmaceutically acceptable salt described herein. Examples of solvates of Compound A include, for example, hydrates. General examples of prodrugs of Compound A, that is to say compounds which break down and/or are metabolized in vivo to provide an active compound, may include esters (e.g., simple esters, or mixed carbonate esters), carbamates, glycosides, ethers, acetals, and ketals.

The respirable dry powders disclosed herein typically comprise 0.25% to about 20% Compound A by weight. In some embodiments, the respirable dry powders contain a low load of Compound A, e.g., 1% or less of Compound A. The Compound A can be in any desired form, such as one or more solid forms that possess high chemical and physical stability for formulation as inhaled medicaments. For example, Compound A can be in solid amorphous form, in solid crystalline form, or in a combination of solid amorphous form and solid crystalline form. In preferred aspects, Compound A is in a crystalline particulate form, such as microparticulate or preferably nanoparticulate as described herein. The dry powders typically further comprise one or more excipients, such as the excipients mannitol and sodium sulfate, which may be present at about 4:1 to about 1:4 (wt %:wt %), or preferably about 1:1 (wt %:wt %), and if desired may include additional excipients. Optionally, other excipients can be used. The dry powder can also include a stabilizer. For example, when the dry powder includes crystalline particulate Compound A, the dry power can also include a stabilizer that is present in a ratio (stabilizer:Compound A) of 1:5 to about 1:20 (wt %:wt %).

As described herein, the inventors discovered that the crystallinity of the Compound A, as well as the size of the Compound A crystalline sub-particles, in addition to the balance of Compound A with other components (stabilizers and excipients) in the dry particles, are important for effective therapy and for reduced toxicity in the lung. As described and exemplified herein, nanocrystalline formulations of Compound A have greatly reduced lung accumulation of Compound A over time, and with reduced toxicity, relative to other formulations such as lactose blend formulations.

This disclosure relates to homogenous respirable dry powders that comprise (e.g., consist of) an active ingredient, a stabilizer and one or more excipients, wherein the active ingredient is Compound A and is present in an amount of about 0.25% to about 20% by weight, the ratio of the stabilizer to the active ingredient is about 1:5 to about 1:20 (wt %:wt %) and the one or more excipients comprise mannitol and sodium sulfate in an amount of about 4:1 to about 1:4 (wt %:wt %), e.g., about 4:1, about 3:1, about 2:1, about 1:1, about 1:2, about 1:3, or about 1:4. Optionally, other excipients can be used.

This disclosure also relates to homogenous respirable dry powders that comprise (e.g., consist of) an active ingredient and one or more excipients, wherein the active ingredient is Compound A and is present in an amount of about 0.25% to about 20% by weight and the one or more excipients comprise mannitol and sodium sulfate in an amount of about 4:1 to about 1:4 (wt %:wt %), e.g., about 4:1, about 3:1, about 2:1, about 1:1, about 1:2, about 1:3, or about 1:4. Optionally, other excipients can be used. The homogenous respirable dry particles of the present disclosure may or may not include a stabilizer.

In some aspects, the respirable dry powder comprising (e.g., consisting of) homogenous respirable dry particles comprise (e.g., consist of) an active ingredient, a stabilizer and one or more excipients.

In other aspects, the respirable dry powder comprising (e.g., consisting of) homogenous respirable dry particles comprise (e.g., consist of) an active ingredient and one or more excipients.

In preferred aspects of the present disclosure, the respirable dry powder comprising (e.g., consisting of) homogenous respirable dry particles comprise (e.g., consist of) an active ingredient, a stabilizer and one or more excipients.

In preferred aspects, the respirable dry particles comprise polysorbate 80 as a stabilizer.

The respirable dry powders of this disclosure can comprise (e.g., consist of) polysorbate 80, one or more excipients, and one or more sub-particles (i.e., particles that are smaller than the respirable dry particle) that comprise crystalline Compound A. The polysorbate 80 is optionally a component of the sub-particle (e.g., incorporated in the sub-particle and/or absorbed to the surface of the sub-particle). Such respirable dry particles can be prepared using any suitable method, such as by preparing a feedstock in which Compound A in crystalline particulate form is suspended in an aqueous solution of excipients, and spray drying the feedstock. Such respirable dry particles can be prepared using any suitable method, such as by preparing a nanoparticle suspension of Compound A in crystalline particulate form suspended in an aqueous solution which contains a stabilizer (such as polysorbate 80) in sufficient amounts to stabilize the suspension. The stabilized nanoparticle suspension can then be added to another solvent (either water or another solvent which is miscible with water and in which, like water, the nanoparticles of crystalline Compound A are poorly soluble) in which the suspension is maintained and one or more excipients is solubilized making the feedstock. This feedstock can then be spray dried to form the respirable dry particles.

The respirable dry powders may be administered to a subject (e.g., a patient) by inhalation, such as oral inhalation. To achieve oral inhalation, a dry powder inhaler may be used, such as a passive dry powder inhaler. The dry powder formulations can be used to treat or prevent inflammatory diseases, for example COPD and/or asthma. The COPD may be stable COPD.

### Definitions

As used herein, the term “about” refers to a relative range of plus or minus 5% of a stated value, e.g., “about 20 mg” would be 20 mg plus or minus 1 mg.

As used herein, the terms “administration” or “administering” of respirable dry particles refers to introducing respirable dry particles to the respiratory tract of a subject.

As used herein, the term “amorphous” indicates lack of significant crystallinity when analyzed via powder X-ray diffraction (XRD). For example, percent crystallinity of Compound A relative to the total amount of compound present in the formulation is less than 50%, less than 40%, less than 30%, less than 20%, less than 10%, or 0% crystalline.

The term “capsule emitted powder mass” or “CEPM” as used herein refers to the amount of respirable dry powder formulation emitted from a capsule or dose unit container during actuation from the respirable dry powder inhaler, such as during an inhalation maneuver. CEPM is measured gravimetrically, typically by weighing a capsule before and after the emission event to determine the mass of powder removed. CEPM can be expressed either as the mass of powder removed, in milligrams, or as a percentage of the initial filled powder mass in the capsule prior to the emission event.

The term “crystalline particulate form” as used herein refers to Compound A (including pharmaceutically acceptable forms thereof including salts, hydrates, stereoisomers and tautomers as the like), that is in the form of a particle (i.e., sub-particle that is smaller than the respirable dry particles that comprise the respirable dry powders disclosed herein) and in which the Compound A is at least about 50% crystalline. The percent crystallinity of Compound A refers to the percentage of the compound that is in crystalline form relative to the total amount of compound present in the sub-particle. If desired, the active ingredient can be at least about 60%, at least about 70%, at least about 80%, at least about 90%, at least about 95%, or about 100% crystalline. Compound A in crystalline particulate form is in the form of a particle that is about 50 nanometers (nm) to about 500 nm volume median diameter (Dv50), preferably 50 nm to 100 nm Dv50, preferably 100 nm to 200 nm Dv50, or preferably 100 nm to about 150 nm.

The term “dispersible” is a term of art that describes the characteristic of a respirable dry powder or respirable dry particles to be dispelled into a respirable aerosol. Dispersibility of a respirable dry powder or respirable dry particles is expressed herein, in one aspect, as the quotient of the volumetric median geometric diameter (VMGD) measured at a dispersion (i.e., regulator) pressure of 1 bar divided by the VMGD measured at a dispersion (i.e., regulator) pressure of 4 bar, or VMGD at 0.5 bar divided by the VMGD at 4 bar as measured by laser diffraction, such as with a HELOS/RODOS. These quotients are referred to herein as “1 bar/4 bar dispersibility ratio” and “0.5 bar/4 bar dispersibility ratio”, respectively, and dispersibility correlates with a low quotient. For example, 1 bar/4 bar dispersibility ratio refers to the VMGD of a dry powder or respirable dry particles emitted from the orifice of a RODOS dry powder disperser (or equivalent technique) at about 1 bar, as measured by a HELOS or other laser diffraction system, divided by the VMGD of the same dry powder or respirable dry particles measured at 4 bar by HELOS/RODOS. Thus, a highly dispersible respirable dry powder or respirable dry particles will have a 1 bar/4 bar dispersibility ratio or 0.5 bar/4 bar dispersibility ratio that is close to 1.0. Highly dispersible powders have a low tendency to agglomerate, aggregate or clump together and/or, if agglomerated, aggregated or clumped together, are easily dispersed or de-agglomerated as they emit from an inhaler and are breathed in by a subject. In another aspect, dispersibility is assessed by measuring the particle size emitted from an inhaler as a function of flowrate. As the flow rate through the inhaler decreases, the amount of energy available to disperse the powder decreases. A highly dispersible powder will have a size distribution such as is characterized aerodynamically by its mass median aerodynamic diameter (MMAD) or geometrically by its VMGD that does not substantially increase over a range of flow rates typical of inhalation by humans, such as about 15 to about 60 liters per minute (LPM), about 20 to about 60 LPM, or about 30 LPM to about 60 LPM. A highly dispersible powder will also have an emitted powder mass or dose, or a capsule emitted powder mass or dose, of about 80% or greater even at the lower inhalation flow rates. VMGD may also be called the volume median diameter (VMD), x50, or Dv50.

The term “dry particles” as used herein refers to respirable particles that may contain up to about 15% total of water and/or another solvent. Preferably, the dry particles contain water and/or another solvent up to about 10% total, up to about 5% total, up to about 1% total, or between 0.01% and 1% total, by weight of the dry particles, or can be substantially free of water and/or other solvent.

The term “dry powder” as used herein refers to compositions that comprise (e.g., consist of) respirable dry particles. A dry powder may contain up to about 15% total of water and/or another solvent. Preferably the dry powder contain water and/or another solvent up to about 10% total, up to about 5% total, up to about 1% total, or between 0.01% and 1% total, by weight of the dry powder, or can be substantially free of water and/or other solvent. In one aspect, the dry powder is a respirable dry powder.

The term “effective amount,” as used herein, refers to the amount of agent needed to achieve the desired effect; such as treating and preventing exacerbations in subjects (e.g., patients) which chronic respiratory disease, such as COPD (including chronic bronchitis and emphysema), e.g., stable COPD, asthma, pediatric asthma, cystic fibrosis, sarcoidosis, idiopathic pulmonary fibrosis, treating and preventing a condition selected from: COPD (including chronic bronchitis and emphysema), e.g., stable COPD, asthma, pediatric asthma, cystic fibrosis, sarcoidosis, idiopathic pulmonary fibrosis, allergic rhinitis, rhinitis, sinusitis, allergic conjunctivitis, conjunctivitis, allergic dermatitis, contact dermatitis, psoriasis, ulcerative colitis, inflamed joints secondary to rheumatoid arthritis or osteoarthritis, rheumatoid arthritis, pancreatitis, cachexia, lung cancer, inhibition of the growth and metastasis of tumors including non-small cell lung carcinoma, breast carcinoma, gastric carcinoma, colorectal carcinomas and malignant melanoma, treating and preventing respiratory viral infections in subjects (e.g., patients) with chronic conditions such as congestive heart failure, diabetes, cancer, or in immunosuppressed subject (e.g., patients), for example post-organ transplant, treating and preventing a condition selected from exacerbation of COPD (e.g., stable COPD) and exacerbation of asthma, treating and preventing virally induced exacerbations of COPD (e.g., stable COPD) and asthma, treating and preventing inflammatory disease in subjects (e.g., patients) with chronic conditions such as selected from a group consisting of congestive heart failure, diabetes, cancer and conditions suffered by immunosuppressed subjects (e.g., patients), and treating or reducing the incidence or severity of an acute exacerbation of a respiratory disease. The actual effective amount for a particular use can vary according to the particular dry powder or respirable dry particle, the mode of administration, and the age, weight, general health of the subject, and severity of the symptoms or condition being treated. Suitable amounts of dry powders and dry particles to be administered, and dosage schedules for a particular subject (e.g., patient) can be determined by a clinician of ordinary skill based on these and other considerations.

As used herein, the term “emitted dose” or “ED” refers to an indication of the delivery of a drug formulation from a suitable inhaler device after a firing or dispersion event. More specifically, for dry powder formulations, the ED is a measure of the percentage of powder that is drawn out of a unit dose package and that exits the mouthpiece of an inhaler device. The ED is defined as the ratio of the drug or powder delivered by an inhaler device to the nominal dose (i.e., the mass of drug or powder per unit dose placed into a suitable inhaler device prior to firing). The ED is an experimentally-measured parameter, and can be determined using the method of USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, MD, 13^{th} Revision, 222-225, 2007. This method utilizes an in vitro device set up to mimic patient dosing. It can also be calculated from the results generated by Next Generation Impactor (NGI) experiments, through summation of all of the drug or powder assayed from the mouthpiece adapter, NGI induction port, and all of the stages within the NGI. The results generated through ED testing per USP 601 and the results generated via the NGI are typically in good agreement.

The term “nominal dose” as used herein refers to an individual dose of Compound A. The nominal dose is the total dose of Compound A within one capsule, blister, or ampule.

The terms “FPF (<X),” “FPF (<X microns),” and “fine particle fraction of less than X microns” as used herein, wherein X equals, for example, 3.4 microns, 4.4 microns, 5.0 microns or 5.6 microns, refer to the fraction of a sample of dry particles that have an aerodynamic diameter of less than X microns. For example, FPF (<X) can be determined by dividing the mass of respirable dry particles deposited on stage two and on the final collection filter of a two-stage collapsed Andersen Cascade Impactor (ACI) by the mass of respirable dry particles weighed into a capsule for delivery to the instrument. This parameter may also be identified as “FPF_TD(<X),” where TD means total dose. A similar measurement can be conducted using an eight-stage ACI. An eight-stage ACI cutoffs are different at the standard 60 L/min flowrate, but the FPF_TD(<X) can be extrapolated from the eight-stage complete data set. The eight-stage ACI result can also be calculated by the USP method of using the dose collected in the ACI instead of what was in the capsule to determine FPF. Similarly, a seven-stage Next Generation Impactor (NGI) can be used.

The terms “FPD (<X)”, ‘FPD <X microns”, FPD(<X microns)” and “fine particle dose of less than X microns” as used herein, wherein X equals, for example, 3.4 microns, 4.4 microns, 5.0 microns or 5.6 microns, refer to the mass of a therapeutic agent delivered by respirable dry particles that have an aerodynamic diameter of less than X micrometers. FPD <X microns can be determined by using an eight-stage ACI at the standard 60 L/min flowrate and summing the mass deposited on the final collection filter, and either directly calculating or extrapolating the FPD value. Similarly, a seven-stage Next Generation Impactor (NGI) can be used.

The term “pharmaceutically acceptable salt” as used herein refers to a salt of a compound prepared with relatively nontoxic acids or bases, depending on the particular substituents found on the respective compound. When compounds of the present disclosure contain relatively acidic functionalities, base addition salts can be obtained by contacting the neutral form of such compounds with a sufficient amount of the desired base, either neat or in a suitable solvent (e.g., an inert solvent). Examples of pharmaceutically acceptable base addition salts include sodium, potassium, calcium, ammonium, organic amino, magnesium salt, or the like. When compounds of the present disclosure contain relatively basic functionalities, acid addition salts can be obtained by contacting the neutral form of such compounds with a sufficient amount of the desired acid, either neat or in a suitable solvent (e.g., an inert solvent). Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfiuric, hydriodic, or phosphorous acids, and the like, as well as the salts derived from organic acids like acetic, propionic, isobutyric, maleic, malonic, benzoic, succinic, suberic, fumaric, lactic, mandelic, pamoic, phthalic, benzenesulfonic, p-toluenesulfonic, citric, tartaric, methanesulfonic acid, and the like. Also included are salts of amino acids such as arginate and the like, and salts of organic acids like glucuronic or galacturonic acids and the like. Certain compounds of the present disclosure can contain both basic and acidic functionalities that allow the compounds to be converted into either base or acid addition salts. These salts may be prepared by methods known to those skilled in the art. Other pharmaceutically acceptable salts known to those of skill in the art are suitable for pharmaceutical compositions the present disclosure relates to.

The term “respirable” as used herein refers to dry particles or dry powders that are suitable for delivery to the respiratory tract (e.g., pulmonary delivery) in a subject by inhalation. Respirable dry powders or dry particles have a mass median aerodynamic diameter (MMAD) of less than about 10 microns, preferably about 5 microns or less.

As used herein, the term “respiratory tract” includes the upper respiratory tract (e.g., nasal passages, nasal cavity, throat, pharynx and larynx), respiratory airways (e.g., trachea, bronchi, and bronchioles) and lungs (e.g., respiratory bronchioles, alveolar ducts, alveolar sacs, and alveoli).

As used herein, the term “lower respiratory tract” includes the respiratory airways (e.g., trachea, bronchi, and bronchioles) and lungs (e.g., respiratory bronchioles, alveolar ducts, alveolar sacs, and alveoli).

The term “small” as used herein to describe respirable dry particles refers to particles that have a volume median geometric diameter (VMGD) of about 10 microns or less, preferably about 5 microns or less, or less than 5 microns.

The term “stabilizer” as used herein refers to a compound that improves the physical stability of Compound A in crystalline particulate form when suspended in a liquid in which the Compound A is poorly soluble (e.g., reduces the aggregation, agglomeration, Ostwald ripening and/or flocculation of the particulates). Suitable stabilizers are surfactants and amphiphilic materials and include Polysorbates (PS; polyoxyethylated sorbitan fatty acid esters; TWEEN®), such as PS20, PS40, PS60 and PS80; fatty acids such as lauric acid, palmitic acid, myristic acid, oleic acid and stearic acid; sorbitan fatty acid esters, such as Span20, Span40, Span60, Span80, and Span 85; phospholipids such as dipalmitoylphosphatidylcholine (DPPC), 1,2-dipalmitoyl-sn-glycero-3-phospho-L-serine (DPPS), 1,2-dipalmitoyl-sn-glycero-3-phosphocholine (DSPC), 1-palmitoyl-2-oleoylphosphatidylcholine (POPC), and 1,2-dioleoyl-sn-glycero-3-phosphocholine (DOPC); phosphatidylglycerols (PGs) such as diphosphatidyl glycerol (DPPG), DSPG, DPPG, POPG, etc.; 1,2-distearoyl-sn-glycero-3-phosphoethanolamine (DSPE); fatty alcohols; benzyl alcohol, polyoxyethylene-9-lauryl ether; glycocholate; surfactin; poloxomers; polyvinylpyrrolidone (PVP); PEG/PPG block co-polymers (Pluronics/Poloxamers); polyoxyethyene chloresteryl ethers; POE alkyl ethers; tyloxapol; lecithin; and the like. Preferred stabilizers are polysorbates and fatty acids. A particularly preferred stabilizer is PS80. In one embodiment, the stabilizer is not a phospholipid.

The term “homogenous respirable dry particle” as used herein refers to particles that each have substantially the same chemical composition. For example, for a formulation that comprises (e.g., consists of) Compound A in crystalline particulate form, polysorbate 80, sodium sulfate and mannitol, the term homogenous dry particle means that substantially every respirable dry particle contains some amount of Compound A in crystalline particulate form, polysorbate 80, sodium sulfate and mannitol.

The term “homogenous respirable dry powder” as used herein refers to dry powders that are composed of a single type of homogenous respirable dry particle. Accordingly, a homogenous respirable dry powder does not include, for example a blend of drug particles with carrier (e.g., lactose carrier) or other excipient particles which do not contain crystalline drug.

### Dry Powders and Dry Particles

The present disclosure relates to dry powder formulations comprising (e.g., consisting of) respirable dry particles that comprise (e.g., consist of) Compound A in crystalline particulate form, polysorbate 80 and one or more excipients, wherein the Compound A is present in an amount of about 0.25% to about 20% by weight, the ratio of the stabilizer to the active ingredient is about 1:5 to about 1:20 (wt %:wt %) and the one or more excipients comprise of mannitol and sodium sulfate in an amount of about 4:1 to about 1:4 (wt %:wt %), e.g., about 4:1, about 3:1, about 2:1, about 1:1, about 1:2, about 1:3, or about 1:4. Optionally, other excipients can be used.

For example, a dry powder formulation disclosed herein can comprise (e.g., consist of) respirable dry particles that comprise (e.g., consist of) Compound A in crystalline particulate form, polysorbate 80, mannitol, and sodium sulfate, wherein Compound A is present in an amount of about 0.25% to about 20% by weight (e.g., between about 1% to about 15% by weight, e.g., between about 5% and about 10% by weight), the ratio of the stabilizer to Compound A is about 1:5 to about 1:20 (wt %:wt %), and the ratio of mannitol to sodium sulfate is about 4:1 to about 1:4 (wt %:wt %), e.g., about 4:1, about 3:1, about 2:1, about 1:1, about 1:2, about 1:3, or about 1:4.

The crystallinity of Compound A, as well as the size of the Compound A sub-particles, among other factors (e.g., choice and amount of excipients and stabilizers), appears to be important for effective therapy and for reduced toxicity in the lung. Without wishing to be bound by any particular theory, it is believed that smaller sub-particles of Compound A in crystalline form will dissolve in the airway lining fluid more rapidly than larger particles of Compound A in the same crystalline form—in part due to the larger amount of surface area. It is also believed that crystalline Compound A will dissolve more slowly in the airway lining fluid than amorphous Compound A for similarly sized particles. Accordingly, the dry powders described herein can be formulated using Compound A in crystalline particulate form that provide for a desired degree of crystallinity and sub-particle size and can be tailored to achieve desired pharmacokinetic properties while avoiding unacceptable toxicity in the lungs.

The respirable dry particles comprise about 0.25% to about 50% Compound A by weight (wt %). It is preferred that the respirable dry particle comprises a suitable amount of Compound A so that a therapeutically effective dose can be administered and maintained without the need to inhale large volumes of dry powder more than three times a day. For example, it is preferred that the respirable dry particles comprise about 0.5% to about 20%, about 1% to about 20%, about 2% to about 20%, about 5% to about 20%, about 2% to about 10%, or about 10% to about 20% Compound A by weight (wt %). For example, respirable dry particles may comprise about 4% to about 6%, or about 9% to about 11% Compound A by weight (wt %). The respirable dry particles may contain about 0.5%, about 1%, about 2%, about 3%, about 4%, about 5%, about 6%, about 7%, about 8%, about 9%, about 10%, about 11%, about 12%, about 13%, about 14%, about 15% or about 20% Compound A by weight (wt %). The amount of Compound A present in the respirable dry particles by weight is also referred to as the “drug load.”

The Compound A may be present in the respirable dry particles in crystalline particulate form (e.g., nano-crystalline or microcrystalline). Preferably, the crystalline particulate form is a sub-particle that is about 50 nm to about 500 nm (Dv50), preferably, with the Compound A being at least 50% crystalline. For example, for any desired drug load, the sub-particle size can be about 50 nm to about 500 nm, about 50 nm to about 150 nm, about 50 to about 100 nm, 100 nm to about 200 nm or 100 nm to about 150 nm (Dv50). In addition, for any desired drug load and sub-particle size, the degree of Compound A crystallinity can be at least about 50%, at least about 60%, at least about 70%, at least about 80%, at least about 90%, at least about 95%, or about 100% crystalline. Preferably, the Compound A is about 100% crystalline.

The Compound A in crystalline particulate form can be prepared in any desired sub-particle size using a suitable method, such as by wet milling, jet milling or other suitable method, and using a stabilizer, such as polysorbate 80, if desired. The respirable dry particles that comprise crystalline Compound A preferably include polysorbate 80 as the preferred stabilizer. The polysorbate 80 helps maintain the desired size of Compound A in crystalline particulate form during wet milling, in spray drying feedstock, and aids in wetting and dispersing and maintaining the physical stability of the Compound A crystalline particulate suspension. It is preferred to use as little polysorbate 80 as is needed to achieve the aforementioned benefits. The amount of polysorbate 80 is typically in a fixed ratio to the amount of Compound A present in the dry particle. For example, the ratio of the stabilizer to the active ingredient (Compound A) is about 1:5 to about 1:20 (wt %:wt % polysorbate 80:Compound A), about 1:8 to about 1:12 (wt %:wt %), about 1:10 to about 1:15 (wt %:wt %) or about 1:15 to about 1:20 (wt %:wt %). Preferably, the ratio of polysorbate 80 to Compound A is about 1:10 (wt %:wt %).

In some embodiments, the amount of polysorbate 80 that is present in the dry particles can be in a range of about 0% to 10% by weight (wt %) or in a range of about 0.2% to about 1% by weight. In particular embodiments, the range is about 1% to less than about 5% by weight, about 0.5% to less than about 5% by weight, about 0.1% to less than about 5% by weight (wt %), or about 0.025% to less than about 5% by weight (wt %). It is generally preferred that the respirable dry particles contain less than about 5% polysorbate 80 by weight (wt %), such as 0.2 wt %, 0.5 wt %, 0.8%, or 1 wt %. It is particularly preferred that respirable dry particles contain about 1.0 wt % polysorbate 80, or 0.5 wt % polysorbate 80.

The respirable dry particles can comprise mannitol and sodium sulfate as excipients, and optionally can also include one or more alternative or additional excipients. Preferably, the dry particles comprise mannitol and sodium sulfate in a ratio of about 4:1 to about 1:4 (wt %:wt %), more preferably about 2:1 to about 1:2 (wt %:wt %), and most preferably about 1:1 (wt %:wt %). If other excipients are included instead of or in addition to mannitol or sodium sulfate, such as sodium chloride, sodium citrate, or leucine, they can also be present in similar ratios as the mannitol and sodium sulfate.

The dry particles can comprise a total excipient content of up to about 98%, up to about 99%, or up to about 99.5% for low drug load particles. The total excipient load can be about 50 wt % to about 99 wt %, about 78 wt % to about 98 wt %, or about 88 wt % to about 98 wt %. Preferred total excipient ranges are from about 78 wt % to about 98 wt %.

Many excipients are well-known in the art and can be included in the dry powders and dry particles described herein. Pharmaceutically acceptable excipients that are particularly preferred for the dry powders and dry particles described herein include monovalent and divalent metal cation salts, carbohydrates, sugar alcohols, and amino acids.

Suitable monovalent metal cation salts, include, for example, sodium salts and potassium salts. Suitable sodium salts that can be present in the respirable dry particles of the present disclosure include, for example, sodium chloride, sodium citrate, sodium sulfate, sodium lactate, sodium acetate, sodium bicarbonate, sodium carbonate, sodium stearate, sodium ascorbate, sodium benzoate, sodium biphosphate, sodium phosphate, sodium bisulfite, sodium borate, sodium gluconate, sodium metasilicate and the like.

Suitable potassium salts include, for example, potassium chloride, potassium bromide, potassium iodide, potassium bicarbonate, potassium nitrite, potassium persulfate, potassium sulfite, potassium bisulfite, potassium phosphate, potassium acetate, potassium citrate, potassium glutamate, dipotassium guanylate, potassium gluconate, potassium malate, potassium ascorbate, potassium sorbate, potassium succinate, potassium sodium tartrate and any combination thereof.

Suitable divalent metal cation salts, include magnesium salts and calcium salts. Suitable magnesium salts include, for example, magnesium lactate, magnesium fluoride, magnesium chloride, magnesium bromide, magnesium iodide, magnesium phosphate, magnesium sulfate, magnesium sulfite, magnesium carbonate, magnesium oxide, magnesium nitrate, magnesium borate, magnesium acetate, magnesium citrate, magnesium gluconate, magnesium maleate, magnesium succinate, magnesium malate, magnesium taurate, magnesium orotate, magnesium glycinate, magnesium naphthenate, magnesium acetylacetonate, magnesium formate, magnesium hydroxide, magnesium stearate, magnesium hexafluorsilicate, magnesium salicylate or any combination thereof.

Suitable calcium salts include, for example, calcium chloride, calcium sulfate, calcium lactate, calcium citrate, calcium carbonate, calcium acetate, calcium phosphate, calcium alginate, calcium stearate, calcium sorbate, calcium gluconate and the like.

A preferred sodium salt is sodium sulfate. A preferred sodium salt is sodium chloride. A preferred sodium salt is sodium citrate. A preferred magnesium salt is magnesium lactate.

Carbohydrate excipients that are useful in this regard include the mono- and polysaccharides, sugar alcohols, dextrans, dextrins, and cyclodextrins, amongst others. Representative monosaccharides include dextrose (anhydrous and the monohydrate; also referred to as glucose and glucose monohydrate), galactose, D-mannose, sorbose and the like. Representative disaccharides include lactose, maltose, sucrose, trehalose and the like. Representative trisaccharides include raffinose and the like. Other carbohydrate excipients including dextran, maltodextrin and cyclodextrins, such as 2-hydroxypropyl-beta-cyclodextrin can be used as desired. A preferred carbohydrate is maltodextrin. Representative sugar alcohols include mannitol, sorbitol and the like. A preferred sugar alcohol is mannitol. Preferred carbohydrates are mannitol, lactose, maltodextrin and trehalose.

Suitable amino acid excipients include any of the naturally occurring amino acids that form a powder under standard pharmaceutical processing techniques and include the non-polar (hydrophobic) amino acids and polar (uncharged, positively charged and negatively charged) amino acids, such amino acids are of pharmaceutical grade and are generally regarded as safe (GRAS) by the U.S. Food and Drug Administration. Representative examples of non-polar amino acids include alanine, isoleucine, leucine, methionine, phenylalanine, proline, tryptophan and valine. Representative examples of polar, uncharged amino acids include cysteine, glycine, glutamine, serine, threonine, and tyrosine. Representative examples of polar, positively charged amino acids include arginine, histidine and lysine. Representative examples of negatively charged amino acids include aspartic acid and glutamic acid. A preferred amino acid is leucine.

In one aspect, the respirable dry particles comprise leucine as one of the one or more excipients in an amount of about 1% to about 9%, about 2% to about 9%, about 3% to about 9%, about 4% to about 9%, about 5% to about 9%, about 1% to about 8%, about 2% to about 8%, about 3% to about 8%, about 4% to about 8%, about 5% to about 8%, about 1% to about 7%, about 2% to about 7%, about 3% to about 7%, about 4% to about 7%, about 5% to about 7%, about 1% to about 6%, about 2% to about 6%, about 3% to about 6%, about 1% to about 5%, about 1%, about 2%, about 3%, about 4%, about 5%, about 6%, about 7%, about 9%, or about 10%.

Preferred dry particles described herein comprise (e.g., consist of) Compound A in crystalline particulate form, polysorbate 80, mannitol, and sodium sulfate. Optionally one or more additional excipients are included. Alternatively, instead of mannitol and sodium sulfate, one or more other excipients are present. If present, the additional or alternative excipient(s) can be a monovalent or divalent metal cation salt, an amino acid, carbohydrate or sugar alcohol. For example, the additional or alternative excipient(s) can be a sodium salt or a magnesium salt, and/or an amino acid (such as leucine). If additional excipients are present, they are present in similar ratios as the mannitol and sodium sulfate. In more particular examples, the additional excipient can be sodium citrate, sodium chloride, magnesium lactate or leucine. Typically, the dry powder formulation does not comprise lactose.

In one aspect, the present disclosure relates to a dry powder formulation comprising (e.g., consisting of) homogenous respirable dry particles that comprise (e.g., consist of) Compound A in a nanocrystalline form (2%), polysorbate 80 (0.2%), mannitol (48.9%) and sodium sulfate (48.9%), where all percentages are by weight of the dry particle (wt %).

In another aspect, the present disclosure relates to a dry powder formulation comprising (e.g., consisting of) homogenous respirable dry particles that comprise (e.g., consist of) Compound A in a nanocrystalline form (5%), polysorbate 80 (0.5%), mannitol (47.25%) and sodium sulfate (47.25%), where all percentages are by weight of the dry particle (wt %).

In another aspect, the present disclosure relates to a dry powder formulation comprising (e.g., consisting of) homogenous respirable dry particles that comprise (e.g., consist of) Compound A in a nanocrystalline form (10%), polysorbate 80 (1.0%), mannitol (44.5%) and sodium sulfate (44.5%), where all percentages are by weight of the dry particle (wt %).

In another aspect, the present disclosure relates to a dry powder formulation comprising (e.g., consisting of) homogenous respirable dry particles that comprise (e.g., consist of) Compound A in a nanocrystalline form (7.6%), polysorbate 80 (0.8%), mannitol (45.8%) and sodium sulfate (45.8%), where all percentages are by weight of the dry particle (wt %).

In another aspect, the present disclosure relates to a dry powder formulation comprising (e.g., consisting of) homogenous respirable dry particles that comprise (e.g., consist of) Compound A in ananocrystalline form (8.0%), polysorbate 80 (0.8%), mannitol (45.6%) and sodium sulfate (45.6%), where all percentages are by weight of the dry particle (wt %).

Table 1 lists exemplary formulations with Compound A, stabilizer, and one or more excipients in a variety of excipient ratios.

TABLE 1

| Formu- | API | Stabilizer | Excipients | Ratio of |
|---|---|---|---|---|
| lation | (wt %) | (wt %) | (wt %) | Excipient |
| I | 5.0% | 0.5% | 94.5% leucine | N/A |
| | Compound A | PS80 | | |
| II | 5.0% | 0.5% | 94.5% mannitol | N/A |
| | Compound A | PS80 | | |
| III | 5.0% | 0.5% | 94.5% sodium chloride | N/A |
| | Compound A | PS80 | | |
| IV | 5.0% | 0.5% | 94.5% sodium sulfate | N/A |
| | Compound A | PS80 | | |
| V | 5.0% | 0.5% | 94.5% trehalose | N/A |
| | Compound A | PS80 | | |
| VI | 50.0% | 5.0% | 45.0% sodium chloride | N/A |
| | Compound A | PS80 | | |
| VII | 2.0% | 0.2% | 48.9% sodium sulfate, | 1:1 |
| | Compound A | PS80 | 48.9% mannitol | |
| VIII | 10.0% | 1.0% | 44.5% sodium sulfate, | 1:1 |
| | Compound A | PS80 | 44.5% mannitol | |
| IX | 2.0% | 0.2% | 48.9% sodium sulfate, | 1:1 |
| | Compound A | PS80 | 48.9% mannitol | |
| X | 5.0% | 0.5% | 47.3% sodium sulfate, | 1:1 |
| | Compound A | PS80 | 47.3% mannitol | |
| XI | 10.0% | 1.0% | 44.5% sodium sulfate, | 1:1 |
| | Compound A | PS80 | 44.5% mannitol | |
| XII | 7.6% | 0.8% | 45.8% sodium sulfate, | 1:1 |
| | Compound A | PS80 | 45.8% mannitol | |
| XIII | 5.0% | n/a | 94.0% Lactohale | 1:1 |
| | Compound A | | LH200, 1.0% | |
| | | | magnesium stearate | |
| XIV | 2.0% | 0.2% | 19.6% sodium sulfate, | 1:4 |
| | Compound A | PS80 | 78.2% mannitol | |
| XV | 2.0% | 0.2% | 78.2% sodium sulfate, | 4:1 |
| | Compound A | PS80 | 19.6% mannitol | |
| XVI | 2.0% | 0.2% | 32.6% sodium sulfate, | 1:2 |
| | Compound A | PS80 | 65.2% mannitol | |
| XVII | 2.0% | 0.2% | 65.2% sodium sulfate, | 2:1 |
| | Compound A | PS80 | 32.6% mannitol | |
| XVIII | 5.0% | 0.5% | 18.9% sodium sulfate, | 1:4 |
| | Compound A | PS80 | 75.6% mannitol | |
| XIX | 5.0% | 0.5% | 75.6% sodium sulfate, | 4:1 |
| | Compound A | PS80 | 18.9% mannitol | |
| XX | 5.0% | 0.5% | 31.5% sodium sulfate, | 1:2 |
| | Compound A | PS80 | 63.0% mannitol | |
| XXI | 5.0% | 0.5% | 63.0% sodium sulfate, | 2:1 |
| | Compound A | PS80 | 31.5% mannitol | |
| XXII | 10.0% | 1.0% | 17.8% sodium sulfate, | 1:4 |
| | Compound A | PS80 | 71.2% mannitol | |
| XXIII | 10.0% | 1.0% | 71.2% sodium sulfate, | 4:1 |
| | Compound A | PS80 | 17.8% mannitol | |
| XXIV | 10.0% | 1.0% | 29.7% sodium sulfate, | 1:2 |
| | Compound A | PS80 | 59.3% mannitol | |
| XXV | 10.0% | 1.0% | 59.3% sodium sulfate, | 2:1 |
| | Compound A | PS80 | 29.7% mannitol | |

In some embodiments, a dry powder disclosed herein is a dry powder of Formulation I. In some embodiments, a dry powder disclosed herein is a dry powder of Formulation II. In some embodiments, a dry powder disclosed herein is a dry powder of Formulation III. In some embodiments, a dry powder disclosed herein is a dry powder of Formulation IV. In some embodiments, a dry powder disclosed herein is a dry powder of Formulation V. In some embodiments, a dry powder disclosed herein is a dry powder of Formulation VI. In some embodiments, a dry powder disclosed herein is a dry powder of Formulation VII. In some embodiments, a dry powder disclosed herein is a dry powder of Formulation VIII. In some embodiments, a dry powder disclosed herein is a dry powder of Formulation IX. In some embodiments, a dry powder disclosed herein is a dry powder of Formulation X. In some embodiments, a dry powder disclosed herein is a dry powder of Formulation XI. In some embodiments, a dry powder disclosed herein is a dry powder of Formulation XII. In some embodiments, a dry powder disclosed herein is a dry powder of Formulation XIII. In some embodiments, a dry powder disclosed herein is a dry powder of Formulation XIV. In some embodiments, a dry powder disclosed herein is a dry powder of Formulation XV. In some embodiments, a dry powder disclosed herein is a dry powder of Formulation XVI. In some embodiments, a dry powder disclosed herein is a dry powder of Formulation XVII. In some embodiments, a dry powder disclosed herein is a dry powder of Formulation XVIII. In some embodiments, a dry powder disclosed herein is a dry powder of Formulation XIX. In some embodiments, a dry powder disclosed herein is a dry powder of Formulation XX. In some embodiments, a dry powder disclosed herein is a dry powder of Formulation XXI. In some embodiments, a dry powder disclosed herein is a dry powder of Formulation XXII. In some embodiments, a dry powder disclosed herein is a dry powder of Formulation XXIII. In some embodiments, a dry powder disclosed herein is a dry powder of Formulation XXIV. In some embodiments, a dry powder disclosed herein is a dry powder of Formulation XV.

The dry powders and/or respirable dry particles are preferably small, mass dense, and dispersible. To measure volumetric median geometric diameter (VMGD), a laser diffraction system may be used, e.g., a Spraytec system (particle size analysis instrument, Malvern Instruments) and a HELOS/RODOS system (laser diffraction sensor with dry dispensing unit, Sympatec GmbH). The respirable dry particles have a VMGD as measured by laser diffraction at the dispersion pressure setting (also called regulator pressure) of 1.0 bar at a maximum orifice ring pressure using a HELOS/RODOS system of about 10 microns or less, about 5 microns or less, about 4 μm or less, about 3 μm or less, about 1 μm to about 5 μm, about 1 μm to about 4 μm, about 1.5 μm to about 3.5 μm, about 2 μm to about 5 μm, about 2 μm to about 4 μm, or about 2 μm to about 3 μm. Preferably, the VMGD is about 5 microns or less, or about 4 μm or less. In one aspect, the dry powders and/or respirable dry particles have a minimum VMGD of about 0.5 microns or about 1.0 micron.

The dry powders and/or respirable dry particles preferably have 1 bar/4 bar dispersibility ratio and/or 0.5 bar/4 bar dispersibility ratio of less than about 2.0 (e.g., about 0.9 to less than about 2), about 1.7 or less (e.g., about 0.9 to about 1.7), about 1.5 or less (e.g., about 0.9 to about 1.5), about 1.4 or less (e.g., about 0.9 to about 1.4), or about 1.3 or less (e.g., about 0.9 to about 1.3), and preferably have a 1 bar/4 bar and/or a 0.5 bar/4 bar of about 1.5 or less (e.g., about 1.0 to about 1.5), and/or about 1.4 or less (e.g., about 1.0 to about 1.4).

The dry powders and/or respirable dry particles preferably have a tap density of at least about 0.2 g/cm³, of at least about 0.25 g/cm³, of at least about 0.3 g/cm³, of at least about 0.35 g/cm³, or of at least 0.4 g/cm³. For example, the dry powders and/or respirable dry particles may have a tap density of greater than 0.4 g/cm³ (e.g., greater than 0.4 g/cm³ to about 1.2 g/cm³), at least about 0.45 g/cm³ (e.g., about 0.45 g/cm³ to about 1.2 g/cm³), at least about 0.5 g/cm³ (e.g., about 0.5 g/cm³ to about 1.2 g/cm³), at least about 0.55 g/cm³ (e.g., about 0.55 g/cm³ to about 1.2 g/cm³), at least about 0.6 g/cm³ (e.g., about 0.6 g/cm³ to about 1.2 g/cm³), or at least about 0.6 g/cm³ to about 1.0 g/cm³. Alternatively, the dry powders and/or respirable dry particles may have a tap density of about 0.01 g/cm³ to about 0.5 g/cm³, about 0.05 g/cm³ to about 0.5 g/cm³, about 0.1 g/cm³ to about 0.5 g/cm³, about 0.1 g/cm³ to about 0.4 g/cm³, or about 0.1 g/cm³ to about 0.4 g/cm³. Alternatively, the dry powders and/or respirable dry particles may have a tap density of about 0.15 g/cm³ to about 1.0 g/cm³. Alternatively, the dry powders and/or respirable dry particles may have a tap density of about 0.3 g/cm³ to about 0.8 g/cm³.

The dry powders and/or respirable dry particles may have a bulk density of at least about 0.1 g/cm³, or at least about 0.8 g/cm³. For example, the dry powders and/or respirable dry particles may have a bulk density of about 0.1 g/cm³ to about 0.6 g/cm³, about 0.2 g/cm³ to about 0.7 g/cm³, about 0.2 g/cm³ to about 0.8 g/cm³, or about 0.3 g/cm³ to about 0.8 g/cm³.

The respirable dry particles, and the dry powders when the dry powders are respirable dry powders, preferably have an MMAD of less than 10 microns, preferably an MMAD of about 5 microns or less, or about 4 microns or less. In one aspect, the respirable dry powders and/or respirable dry particles preferably have a minimum MMAD of about 0.5 microns, or about 1.0 micron. In one aspect, the respirable dry powders and/or respirable dry particles preferably have a minimum MMAD of about 2.0 microns, about 3.0 microns, or about 4.0 microns. In another aspect, the respirable dry powders and/or respirable dry particles preferably have an MMAD of about 1.0 micron to about 5.0 microns.

The dry powders and/or respirable dry particles preferably have an FPF of less than about 5.6 microns (FPF<5.6 μm) of the total dose of at least about 35%, preferably at least about 45%, at least about 60%, between about 45% to about 80%, or between about 60% and about 80%.

The dry powders and/or respirable dry particles preferably have an FPF of less than about 3.4 microns (FPF<3.4 μm) of the total dose of at least about 20%, preferably at least about 25%, at least about 30%, at least about 40%, between about 25% and about 60%, or between about 40% and about 60%.

The dry powders and/or respirable dry particles preferably have a total water and/or solvent content of up to about 15% by weight, up to about 10% by weight, up to about 5% by weight, up to about 1%, or between about 0.01% and about 1%, or may be substantially free of water or other solvent.

The dry powders and/or respirable dry particles preferably may be administered with low inhalation energy. In order to relate the dispersion of powder at different inhalation flow rates, volumes, and from inhalers of different resistances, the energy required to perform the inhalation maneuver may be calculated. Inhalation energy can be calculated from the equation E=R²Q²V where E is the inhalation energy in Joules, R is the inhaler resistance in kPa^{1/2}/LPM, Q is the steady flow rate in L/min and V is the inhaled air volume in L.

Healthy adult populations are predicted to be able to achieve inhalation energies ranging from 2.9 Joules for comfortable inhalations to 22 Joules for maximum inhalations by using values of peak inspiratory flow rate (PIFR) measured by Clarke et al. (Journal of Aerosol Med, 6(2), p. 99-110, 1993) for the flow rate Q from two inhaler resistances of 0.02 and 0.055 kPa^{1/2}/LPM, with an inhalation volume of 2 L based on both FDA guidance documents for dry powder inhalers and on the work of Tiddens et al. (Journal of Aerosol Med, 19(4), p. 456-465, 2006) who found adults averaging 2.2 L inhaled volume through a variety of DPIs.

Mild, moderate and severe adult COPD patients are predicted to be able to achieve maximum inhalation energies of 5.1 to 21 Joules, 5.2 to 19 Joules, and 2.3 to 18 Joules respectively. This is again based on using measured PIFR values for the flow rate Q in the equation for inhalation energy. The PIFR achievable for each group is a function of the inhaler resistance that is being inhaled through. The work of Broeders et al. (Eur. Respir. J., 18, p. 780-783, 2001) was used to predict maximum and minimum achievable PIFR through two dry powder inhalers of resistances 0.021 and 0.032 kPa^{1/2}/LPM for each.

Similarly, adult asthmatic patients are predicted to be able to achieve maximum inhalation energies of 7.4 to 21 Joules based on the same assumptions as the COPD population and PIFR data from Broeders et al. (supra).

Healthy adults and children, COPD patients, asthmatic patients ages 5 and above, and CF patients, for example, are capable of providing sufficient inhalation energy to empty and disperse the dry powder formulations of the present disclosure.

The dry powders and/or respirable dry particles are preferably characterized by a high emitted dose, such as a CEPM of at least 75%, at least 80%, at least 85%, at least 90%, or at least 95%, from a passive dry powder inhaler subject to a total inhalation energy of about 5 Joules, about 3.5 Joules, about 2.4 Joules, about 2 Joules, about 1 Joule, about 0.8 Joules, about 0.5 Joules, or about 0.3 Joules applied to the dry powder inhaler. The receptacle holding the dry powders and/or respirable dry particles may contain about 5 mg, about 7.5 mg, about 10 mg, about 15 mg, about 20 mg, or about 30 mg. In one aspect, the dry powders and/or respirable dry particles are characterized by a CEPM of 80% or greater, and a VMGD of 5 microns or less when emitted from a passive dry powder inhaler having a resistance of about 0.036 sqrt(kPa)/liters per minute under the following conditions: an air flow rate of 30 LPM, run for 3 seconds using a size 3 capsule that contains a total mass of 10 mg. In another aspect, the dry powders and/or respirable dry particles are characterized by a CEPM of 80% or greater, and a VMGD of 5 microns or less when emitted from a passive dry powder inhaler having a resistance of about 0.036 sqrt(kPa)/liters per minute under the following conditions: an air flow rate of 20 LPM, run for 3 seconds using a size 3 capsule that contains a total mass of 10 mg. In a further aspect, the dry powders and/or respirable dry particles are characterized by a CEPM of 80% or greater, and a VMGD of 5 microns or less when emitted from a passive dry powder inhaler having a resistance of about 0.036 sqrt(kPa)/liters per minute under the following conditions: an air flow rate of 15 LPM, run for 4 seconds using a size 3 capsule that contains a total mass of 10 mg.

The dry powder can fill the unit dose container, or the unit dose container can be at least 2% full, at least 5% full, at least 10% full, at least 20% full, at least 30% full, at least 40% full, at least 50% full, at least 60% full, at least 70% full, at least 80% full, or at least 90% full. The unit dose container can be a capsule (e.g., size 000, 00, 0E, 0, 1, 2, 3, and 4, with respective volumetric capacities of 1.37 mL, 950 μL, 770 μL, 680 μL, 480 μL, 360 μL, 270 μL, and 200 μL). The capsule can be at least about 2% full, at least about 5% full, at least about 10% full, at least about 20% full, at least about 30% full, at least about 40% full, or at least about 50% full. The unit dose container can be a blister. The blister can be packaged as a single blister or as part of a set of blisters, for example, 7 blisters, 14 blisters, 28 blisters or 30 blisters. The one or more blister can be preferably at least 30% full, at least 50% full or at least 70% full.

An advantage of the present disclosure is the production of powders that disperse well across a wide range of flow rates and are relatively flowrate independent. The dry powders and/or respirable dry particles of the present disclosure enable the use of a simple, passive DPI for a wide patient population.

In particular aspects, the present disclosure relates to dry powders and/or respirable dry particles that comprise Compound A in crystalline particulate form (e.g., particles of about 50 nm to about 500 nm (Dv50), about 50 nm to about 150 nm, about 50 to about 100 nm, about 100 nm to about 200 nm, or about 100 nm to about 150 nm), a stabilizer, and optionally one or more excipients.

The dry powders and/or respirable dry particles are preferably small, mass dense, and dispersible. To measure volumetric median geometric diameter (VMGD), a laser diffraction system may be used, e.g., a Spraytec system (particle size analysis instrument, Malvern Instruments) and a HELOS/RODOS system (laser diffraction sensor with dry dispensing unit, Sympatec GmbH). The respirable dry particles have a VMGD as measured by laser diffraction at the dispersion pressure setting (also called regulator pressure) of 1.0 bar at a maximum orifice ring pressure using a HELOS/RODOS system of about 10 microns or less, about 5 microns or less, about 4 μm or less, about 3 μm or less, about 1 μm to about 5 μm, about 1 μm to about 4 μm, about 1.5 μm to about 3.5 μm, about 2 μm to about 5 μm, about 2 μm to about 4 μm, or about 2 μm to about 3 μm. Preferably, the VMGD is about 5 microns or less, or about 4 μm or less. In one aspect, the dry powders and/or respirable dry particles have a minimum VMGD of about 0.5 microns or about 1.0 micron.

The dry powders and/or respirable dry particles described by any of the ranges or specifically disclosed formulations, characterized in the previous paragraph, may be filled into a receptacle, for example a capsule or a blister. When the receptacle is a capsule, the capsule is, for example, a size 2 or a size 3 capsule, and is preferably a size 3 capsule. The capsule material may be, for example, gelatin or HPMC (hydroxypropyl methylcellulose), and is preferably HPMC.

The dry powder and/or respirable dry particles described and characterized above may be contained in a dry powder inhaler (DPI). The DPI may be a capsule-based DPI or a blister-based DPI, and is preferably a capsule-based DPI. More preferably, the dry powder inhaler is selected from the RS01 family of dry powder inhalers (Plastiape S.p.A., Italy). More preferably, the dry powder inhaler is selected from the RS01 HR or the RS01 UHR2. Most preferably, the dry powder inhaler is the RS01 HR.

### Methods for Preparing Dry Powders and Dry Particles

The respirable dry particles and dry powders can be prepared using any suitable method, with the proviso that the dry powder formulation cannot be an extemporaneous dispersion. Many suitable methods for preparing dry powders and/or respirable dry particles are conventional in the art, and include single and double emulsion solvent evaporation, spray drying, spray-freeze drying, milling (e.g., jet milling), blending, solvent extraction, solvent evaporation, phase separation, simple and complex coacervation, interfacial polymerization, suitable methods that involve the use of supercritical carbon dioxide (CO₂), sonocrystallization, nanoparticle aggregate formation and other suitable methods, including combinations thereof. Respirable dry particles can be made using methods for making microspheres or microcapsules known in the art. These methods can be employed under conditions that result in the formation of respirable dry particles with desired aerodynamic properties (e.g., aerodynamic diameter and geometric diameter). If desired, respirable dry particles with desired properties, such as size and density, can be selected using suitable methods, such as sieving.

Suitable methods for selecting respirable dry particles with desired properties, such as size and density, include wet sieving, dry sieving, and aerodynamic classifiers (such as cyclones).

The respirable dry particles are preferably spray dried. Suitable spray-drying techniques are described, for example, by K. Masters in “Spray Drying Handbook”, John Wiley & Sons, New York (1984). Generally, during spray-drying, heat from a hot gas such as heated air or nitrogen is used to evaporate a solvent from droplets formed by atomizing a continuous liquid feed. When hot air is used, the moisture in the air is at least partially removed before its use. When nitrogen is used, the nitrogen gas can be run “dry”, meaning that no additional water vapor is combined with the gas. If desired the moisture level of the nitrogen or air can be set before the beginning of spray dry run at a fixed value above “dry” nitrogen. If desired, the spray drying or other instruments, e.g., jet milling instrument, used to prepare the dry particles can include an inline geometric particle sizer that determines a geometric diameter of the respirable dry particles as they are being produced, and/or an inline aerodynamic particle sizer that determines the aerodynamic diameter of the respirable dry particles as they are being produced.

For spray drying, solutions, emulsions or suspensions that contain the components of the dry particles to be produced in a suitable solvent (e.g., aqueous solvent, organic solvent, aqueous-organic mixture or emulsion) are distributed to a drying vessel via an atomization device. For example, a nozzle or a rotary atomizer may be used to distribute the solution or suspension to the drying vessel. The nozzle can be a two-fluid nozzle, which can be in an internal mixing setup or an external mixing setup. Alternatively, a rotary atomizer having a 4- or 24-vaned wheel may be used. Examples of suitable spray dryers that can be outfitted with a rotary atomizer and/or a nozzle, include, a Mobile Minor Spray Dryer or the Model PSD-1, both manufactured by GEA Niro, Inc. (Denmark), Büchi B-290 Mini Spray Dryer (BÜCHI Labortechnik AG, Flawil, Switzerland), ProCepT Formatrix R&D spray dryer (ProCepT nv, Zelzate, Belgium), among several other spray dryer options. Actual spray drying conditions will vary depending, in part, on the composition of the spray drying solution or suspension and material flow rates. The person of ordinary skill will be able to determine appropriate conditions based on the compositions of the solution, emulsion or suspension to be spray dried, the desired particle properties and other factors. In general, the inlet temperature to the spray dryer is about 90° C. to about 300° C. The spray dryer outlet temperature will vary depending upon such factors as the feed temperature and the properties of the materials being dried. Generally, the outlet temperature is about 50° C. to about 150° C. If desired, the respirable dry particles that are produced can be fractionated by volumetric size, for example, using a sieve, or fractioned by aerodynamic size, for example, using a cyclone, and/or further separated according to density using techniques known to those of skill in the art.

To prepare the respirable dry particles of the present disclosure, generally, an emulsion or suspension that contains the desired components of the dry powder (i.e., a feedstock) is prepared and spray dried under suitable conditions. Preferably, the dissolved or suspended solids concentration in the feedstock is at least about 1 g/L, at least about 2 g/L, at least about 5 g/L, at least about 10 g/L, at least about 15 g/L, at least about 20 g/L, at least about 30 g/L, at least about 40 g/L, at least about 50 g/L, at least about 60 g/L, at least about 70 g/L, at least about 80 g/L, at least about 90 g/L or at least about 100 g/L. The feedstock can be provided by preparing a single solution, suspension or emulsion by dissolving, suspending, or emulsifying suitable components (e.g., salts, excipients, other active ingredients) in a suitable solvent. The solution, emulsion or suspension can be prepared using any suitable methods, such as bulk mixing of dry and/or liquid components or static mixing of liquid components to form a combination. For example, a hydrophilic component (e.g., an aqueous solution) and a hydrophobic component (e.g., an organic solution) can be combined using a static mixer to form a combination. The combination can then be atomized to produce droplets, which are dried to form respirable dry particles. Preferably, the atomizing step is performed immediately after the components are combined in the static mixer. Alternatively, the atomizing step is performed on a bulk mixed solution.

The feedstock can be prepared using any solvent in which the active ingredient in particulate form has low solubility, such as an organic solvent, an aqueous solvent or mixtures thereof. Suitable organic solvents that can be employed include but are not limited to alcohols such as, for example, ethanol, methanol, propanol, isopropanol, butanols, and others. Other organic solvents include but are not limited to tetrahydrofuran (THF), perfluorocarbons, dichloromethane, chloroform, ether, ethyl acetate, methyl tert-butyl ether and others. Co-solvents that can be employed include an aqueous solvent and an organic solvent, such as, but not limited to, the organic solvents as described above. Aqueous solvents include water and buffered solutions. A preferred solvent is water.

Various methods (e.g., static mixing, bulk mixing) can be used for mixing the solutes and solvents to prepare feedstocks, which are known in the art. If desired, other suitable methods of mixing may be used. For example, additional components that cause or facilitate the mixing can be included in the feedstock. For example, carbon dioxide produces fizzing or effervescence and thus can serve to promote physical mixing of the solute and solvents.

The feedstock or components of the feedstock can have any desired pH, viscosity or other properties. If desired, a pH buffer can be added to the solvent or co-solvent or to the formed mixture. Generally, the pH of the mixture ranges from about 3 to about 8.

Dry powder and/or respirable dry particles can be fabricated and then separated, for example, by filtration or centrifugation by means of a cyclone, to provide a particle sample with a preselected size distribution. For example, greater than about 30%, greater than about 40%, greater than about 50%, greater than about 60%, greater than about 70%, greater than about 80%, or greater than about 90% of the respirable dry particles in a sample can have a diameter within a selected range. The selected range within which a certain percentage of the respirable dry particles fall can be, for example, any of the size ranges described herein, such as between about 0.1 to about 3 microns VMGD.

The suspension may be a nano-suspension, similar to an intermediate for making dry powder containing nano-crystalline drug.

The dry powder may be a drug embedded in a matrix material, such as sodium sulfate and mannitol. Optionally, the dry powder may be spray dried such that the dry particles are small, dense, and dispersible.

The dry powders can consist solely of the respirable dry particles described herein without other carrier or excipient particles (referred to as “neat powders”).

In a preferred embodiment, the dry powders do not contain carrier particles. In one aspect, the crystalline particles of Compound A are embedded in a matrix comprising excipient and/or stabilizer. The dry powder may comprise respirable dry particles of uniform content, wherein each particle contains crystalline Compound A.

The dry powders can comprise respirable dry particles wherein at least 98%, at least 99%, or substantially all of the particles (by weight) contain Compound A.

The dry powders can comprise crystalline particles of Compound A distributed throughout a matrix comprising one or more excipients. The excipients can comprise any number of salts, sugars, lipids, amino acids, surfactants, polymers, or other components suitable for pharmaceutical use. Preferred excipients include sodium sulfate and mannitol. The dry powders are typically manufactured by first processing the crystalline Compound A to adjust the particle size using any number of techniques that are familiar to those of skill in the art (e.g., wet milling, jet milling). The crystalline Compound A is processed in an antisolvent with polysorbate 80 to form a suspension. The stabilized suspension of crystalline Compound A is then spray dried with the one or more additional excipients. The resulting dry particles comprise crystalline Compound A dispersed throughout an excipient matrix with each dry particle having a homogenous composition.

In a particular embodiment, a dry powder of the present disclosure is made by starting with crystalline Compound A, which is usually obtainable in a micro-crystalline size range. The particle size of the micro-crystalline Compound A is reduced into the nano-crystalline size using any of a number of techniques familiar to those of skill in the art, including but not limited to, high-pressure homogenization, high-shear homogenization, jet-milling, pin milling, microfluidization, or wet milling (also known as ball milling, pearl milling or bead milling). Wet milling is often preferred, as it is able to achieve a wide range of particle size distributions, including those in the nanometer (<1 μm) size domain. What becomes especially important in the sub-micron size domain is the use of surface stabilizing components, such as surfactants (e.g., polysorbate 80, also called Tween 80). Polysorbate 80 enables the creation of submicron particles during milling and the formation of physically stable suspensions, as they sequester the many high energy surfaces created during milling preventing aggregation and sedimentation. Thus, the presence of the polysorbate 80 is important to spray drying homogenous micro-particles as the polysorbate 80 allows for the formation of a uniform and stable suspension ensuring compositional homogeneity across particles. The use of polysorbate 80 allows for formation of micro-suspensions or nano-suspensions. With the polysorbate 80, the nano-crystalline Compound A particles are suspended in a stable colloidal suspension in the anti-solvent. The anti-solvent for the Compound A can utilize water, or a combination of water and other miscible solvents such as alcohols or ketones as the continuous anti-solvent phase for the colloidal suspension. A spray drying feedstock may be prepared by dissolving the soluble components in a desired solvent(s) followed by dispersing the polysorbate 80-stabilized crystalline Compound A nanosuspension in the resulting feedstock while mixing, although the process is not limited to this specific order of operations.

In some embodiments, variations of dry powders described herein are made by maintaining the amount of Compound A, while reducing the amount of surfactant. In yet other embodiments, variations of the dry powders described herein are made by increasing the amount of Compound A, while maintaining the original amount of surfactant.

Methods for analyzing the dry powders and/or respirable dry particles are found in the Exemplification section below.

### Therapeutic Use and Methods

The dry powders and/or respirable dry particles of the present disclosure are particularly suitable for topical delivery, such as topical delivery to the lungs, in particular for the treatment of respiratory disease, for example chronic respiratory diseases such as COPD (e.g., stable COPD) and/or asthma.

The dry powders and/or respirable dry particles of the present disclosure are particularly suitable for use in the treatment and/or prophylaxis of exacerbations of inflammatory diseases, in particular viral exacerbations, in subjects (e.g., patients) with one or more of the following chronic conditions such as congestive heart failure, COPD (e.g., stable COPD), asthma, diabetes, cancer and/or in immunosuppressed subjects (e.g., patients), for example post-organ transplant. In some embodiments, the dry powders disclosed herein are used to treat COPD, e.g., stable COPD.

In another aspect of the present disclosure, it may be useful in the treatment of one or more respiratory disorders including COPD (including chronic bronchitis and emphysema), e.g., stable COPD, asthma, pediatric asthma, cystic fibrosis, sarcoidosis, idiopathic pulmonary fibrosis, allergic rhinitis, rhinitis, sinusitis, especially asthma, and COPD (including chronic bronchitis and emphysema), e.g., stable COPD. In some embodiments, the dry powders disclosed herein are used to treat acute exacerbations of COPD. In some embodiments, the dry powders disclosed herein are used to treat acute exacerbations of stable COPD.

In other embodiments, the dry powders disclosed herein are used to treat lung cancer.

The present disclosure may also be useful in the treatment of one or more conditions which may be treated by topical or local therapy including allergic conjunctivitis, conjunctivitis, allergic dermatitis, contact dermatitis, psoriasis, ulcerative colitis, inflamed joints secondary to rheumatoid arthritis or to osteoarthritis.

It is also expected that the dry powders and/or respirable dry particles of the present disclosure may be useful in the treatment of certain other conditions including rheumatoid arthritis, pancreatitis, cachexia, lung cancer, inhibition of the growth and metastasis of tumors including non-small cell lung carcinoma, breast carcinoma, gastric carcinoma, colorectal carcinomas and malignant melanoma.

In some aspects, the present disclosure may be useful in the treatment of eye diseases or disorders including allergic conjunctivitis, conjunctivitis, diabetic retinopathy, macular oedema (including wet macular oedema and dry macular oedema), post-operative cataract inflammation or, particularly, uveitis (including posterior, anterior and pan uveitis).

In other aspects, the present disclosure may be useful in the treatment of gastrointestinal diseases or disorders including ulcerative colitis or Crohn's disease.

In another aspect, the present disclosure may also re-sensitize the subject's (e.g., patient's) condition to treatment with a corticosteroid, when the subject's condition has become refractory to such treatment regimens.

In some aspects, the present disclosure may also be useful for the treatment of rheumatoid arthritis.

In other aspects, Compound A may have antiviral properties, for example the ability to prevent infection of cells (such as respiratory epithelial cells) with a picomavirus, in particular a rhinovirus, influenza or respiratory syncytial virus. Thus the active ingredient is thought to be an antiviral agent, in particular suitable for the prevention, treatment or amelioration of picomavirus infections, such as rhinovirus infection, influenza or respiratory syncytial virus.

The dry powders and/or respirable dry particles can be administered to the respiratory tract of a subject in need thereof using any suitable method, such as instillation techniques, and/or an inhalation device, such as a dry powder inhaler (DPI) or metered dose inhaler (MDI). A number of DPIs are available, such as, the inhalers disclosed is U.S. Pat. Nos. 4,995,385 and 4,069,819, SPINHALER® (Fisons, Loughborough, U.K.), ROTAHALERS®, DISKHALER® and DISKUS® (GlaxoSmithKline, Research Triangle Technology Park, North Carolina), FLOWCAPS® (Hovione, Loures, Portugal), INHALATORS® (Boehringer-Ingelheim, Germany), AEROLIZER® (Novartis, Switzerland), high-resistance, ultrahigh-resistance and low-resistance RS01 (Plastiape, Italy) and others known to those skilled in the art.

The following scientific journal articles are incorporated by reference for their thorough overview of the following dry powder inhaler (DPI) configurations: 1) Single-dose Capsule DPI, 2) Multi-dose Blister DPI, and 3) Multi-dose Reservoir DPI. N. Islam, E. Gladki, “Dry powder inhalers (DPIs)—A review of device reliability and innovation”, International Journal of Pharmaceuticals, 360(2008):1-11. H. Chystyn, “Diskus Review”, International Journal of Clinical Practice, June 2007, 61, 6, 1022-1036. H. Steckel, B. Muller, “In vitro evaluation of dry powder inhalers I: drug deposition of commonly used devices”, International Journal of Pharmaceuticals, 154(1997):19-29. Some representative capsule-based DPI units are RS-01 (Plastiape, Italy), TURBOSPIN® (PH&T, Italy), BREZHALER® (Novartis, Switzerland), AEROLIZER (Novartis, Switzerland), PODHALER® (Novartis, Switzerland), HANDIHALER® (Boehringer Ingelheim, Germany), AIR® (Civitas, Massachusetts), DOSE ONE® (Dose One, Maine), and ECLIPSE® (Rhone Poulenc Rorer). Some representative unit dose DPIs are CONIX® (3M, Minnesota), CRICKET® (Mannkind, California), DREAMBOAT® (Mannkind, California), OCCORIS® (Team Consulting, Cambridge, UK), SOLIS® (Sandoz), TRIVAIR® (Trimel Biopharma, Canada), TWINCAPS® (Hovione, Loures, Portugal). Some representative blister-based DPI units are DISKUS (GlaxoSmithKline (GSK), UK), DISKHALER® (GSK), TAPER DRY® (3M, Minnesota), GEMINI® (GSK), TWINCER® (University of Groningen, Netherlands), ASPIRAIR (Vectura, UK), ACU-BREATHE® (Respirics, Minnisota, USA), EXUBRA® (Novartis, Switzerland), GYROHALER® (Vectura, UK), OMNIHALER® (Vectura, UK), MICRODOSE® (Microdose Therapeutix, USA), MULTIHALER® (Cipla, India) PROHALER® (Aptar), TECHNOHALER® (Vectura, UK), and XCELOVAIR® (Mylan, Pennsylvania). Some representative reservoir-based DPI units are CLICKHALER® (Vectura), NEXT DPI® (Chiesi), EASYHALER® (Orion), NOVOLIZER® (Meda), PULMOJET® (sanofi-aventis), PULVINAL® (Chiesi), SKYEHALER® (Skyepharma), DUOHALER® (Vectura), TAIFUN® (Akela), FLEXHALER® (AstraZeneca, Sweden), TURBUHALER® (AstraZeneca, Sweden), and TWISTHALER® (Merck), and others known to those skilled in the art.

Generally, inhalation devices (e.g., DPIs) are able to deliver a maximum amount of dry powder or dry particles in a single inhalation, which is related to the capacity of the blisters, capsules (e.g., size 000, 00, 0E, 0, 1, 2, 3 and 4, with respective volumetric capacities of 1.37 mL, 950 μL, 770 μL, 680 μL, 480 μL, 360 μL, 270 μL and 200 μL) or other means that contain the dry powders and/or respirable dry particles within the inhaler. Preferably, the blister has a volume of about 360 microliters or less, about 270 microliters or less, or more preferably, about 200 microliters or less, about 150 microliters or less, or about 100 microliters or less. Preferably, the capsule is a size 2 capsule, or a size 4 capsule. More preferably, the capsule is a size 3 capsule. Accordingly, delivery of a desired dose or effective amount may require two or more inhalations. Preferably, each dose that is administered to a subject in need thereof contains an effective amount of respirable dry particles or dry powder and is administered using no more than about 4 inhalations. For example, each dose of dry powder or respirable dry particles can be administered in a single inhalation or 2, 3, or 4 inhalations. The dry powders and/or respirable dry particles are preferably administered in a single, breath-activated step using a passive DPI. When this type of device is used, the energy of the subject's inhalation both disperses the respirable dry particles and draws them into the respiratory tract.

The amount of a dry powder, dry particles, or composition administered to achieve a therapeutic effect will vary from subject to subject, depending on various factors including, e.g., species, age, and general condition of the subject; severity of the disease or disorder being treated (e.g., COPD or asthma) or its side-effects; the identity of the particular dry powder, dry particles, or composition; mode of administration, and the like. The desired dosage may be delivered (e.g., by inhalation) one or more times a day, e.g., once daily, twice daily, thrice daily, etc., or may be delivered less frequently, e.g., every other day, every third day, every week, every two weeks, every three weeks, monthly, etc. In some embodiments, the dry powder is administered once daily. The desired dosage may be delivered on an as needed basis. The desired dosage may also be delivered using multiple administrations (e.g., inhalations), e.g., two, three, four, five, or more administrations.

The dry powders, dry particles, and compositions disclosed herein may be administered to a subject repeatedly over a period of time (e.g., chronically). For example, a dry powder, dry particles, or a composition disclosed herein may be administered once daily, twice daily, thrice daily or more, over a period of multiple days, multiple weeks, multiple months, or multiple years, e.g., over a period of 2 days, 3 days, 4 days, 5 days, 6 days, 7 days, 8 days, 10 days, 14 days, 20 days, 28 days, 30 days, 2 weeks, 3 weeks, 4 weeks, 2 months, 3 months, 4 months, 5 months, 6 months, 8 months, 10 months, 1 year, 1.5 years, 2 years, 2.5 years, 5 years, 10 years, or longer.

Without wishing to be bound by theory, it is believed that repeated administration of a non-lactose blend formulation disclosed herein does not lead to significant accumulation (plasma accumulation, lung accumulation, or both). For example, a dry powder, dry particles, or composition disclosed herein, that is not a lactose blend, may have less than 6-fold plasma accumulation, e.g., 5-fold, 4-fold, 3-fold, 2-fold, or less plasma accumulation. A dry powder, dry particles, or composition disclosed herein, that is not a lactose blend, may have less than 6-fold lung accumulation, e.g., 5-fold, 4-fold, 3-fold, 2-fold, or less lung accumulation.

Without wishing to be bound by any theory, a dry powder, dry particles, or composition disclosed herein that is not a lactose blend may result in less accumulation (e.g., lung accumulation or serum accumulation), as compared to a composition that is a lactose blend (e.g., Formulation XIII) after repeated dosing (e.g., repeated daily dosing for a period of 2 or more days, e.g., after 4, 6, 8, 12, 14, 16, 18, 20, 24, or 28 days, or longer of daily dosing).

Additionally, without wishing to be bound by theory, it is believed that plasma accumulation increases with increasing dose duration when using lactose-blend formulations, but not when using a non-lactose blend formulation disclosed herein. It is further believed that administering a non-lactose blend formulation disclosed herein leads to relatively consistent accumulation, e.g., up to 6 or 9 months of dosing, or longer. Additionally, it is believed that clearance from the plasma after the end of dosing is generally faster when using a non-lactose blend formulation disclosed herein, compared to a lactose-blend formulation (e.g., Formulation XIII). Further, without wishing to be bound by theory, it is believed that lactose blend formulations of Compound A can have poorly controlled exposure kinetics, which can result in accumulation that increases with increasing duration of dosing, and exhibits slow clearance; and the non-lactose blend formulations disclosed herein, on the other hand, have less overall accumulation and reach steady state and clear more quickly, allowing for more controlled and predictable exposure regardless of dose duration.

Administering a dry powder, dry particles, or a composition disclosed herein to a subject may result in a measurable pharmacodynamic effect, e.g., as measured by one or more biomarkers, indicating efficacy and/or target engagement of the active agent (e.g., Compound A) in the dry powder, dry particles, or composition. For example, a dry powder, dry particle, or a composition disclosed herein may reduce inflammatory cells (e.g., neutrophils), e.g., within the lungs or sputum, when administered to a subject. In some embodiments, administering a dry powder, dry particles, or a composition disclosed herein to a subject results in a reduction in sputum neutrophils. Also, a dry powder, dry particle, or a composition disclosed herein may reduce the phosphorylation of p38 mitogen activated protein kinase (MAPK), a potent mediator of inflammation, when administered to a subject. In some embodiments, administering a dry powder, dry particles, or a composition disclosed herein to a subject results in a reduction in phosphorylation of p38 MAPK. The magnitude of observed effects of administering a dry powder, dry particles, or a composition disclosed herein may increase with dose.

Dry powders and/or respirable dry particles suitable for use in the methods of the present disclosure can travel through the upper airways (i.e., the oropharynx and larynx), the lower airways, which include the trachea followed by bifurcations into the bronchi and bronchioli, and through the terminal bronchioli which in turn divide into respiratory bronchioli leading then to the ultimate respiratory zone, the alveoli or the deep lung. In one embodiment of the present disclosure, most of the mass of respirable dry particles deposit in the deep lung. In another embodiment of the present disclosure, delivery is primarily to the central airways. In another embodiment, delivery is to the upper airways. In a preferred embodiment, most of the mass of the respirable dry particles deposit in the conducting airways.

If desired or indicated, the dry powders and respirable dry particles described herein can be administered with one or more other therapeutic agents. The other therapeutic agents can be administered by any suitable route, such as orally, parenterally (e.g., intravenous, intra-arterial, intramuscular, or subcutaneous injection), topically, by inhalation (e.g., intrabronchial, intranasal or oral inhalation, intranasal drops), rectally, vaginally, and the like. The respirable dry particles and dry powders can be administered before, substantially concurrently with, or subsequent to administration of the other therapeutic agent. Preferably, the dry powders and/or respirable dry particles and the other therapeutic agent are administered so as to provide substantial overlap of their pharmacologic activities.

The dry powders and respirable dry particles described herein are intended to be inhaled as such, and the present disclosure excludes the use of the dry powder formulation in making an extemporaneous dispersion. An extemporaneous dispersion is known by those skilled in the art as a preparation completed just before use, which means right before the administration of the drug to the subject (e.g., patient). As used herein, the term “extemporaneous dispersion” refers to all of the cases in which the solution or suspension is not directly produced by the pharmaceutical industry and commercialized in a ready to be used form, but is prepared in a moment that follows the preparation of the dry solid composition, usually in a moment close to the administration to the subject (e.g., patient).

Administering a dry powder disclosed herein to the respiratory tract of a subject, at a nominal dose of about 500 μg, may result in a systemic Cₘₐₓ of Compound A of about 150-200 μg/mL, e.g., about 160 μg/mL, about 170 μg/mL, or about 180 μg/mL. Administering a dry powder disclosed herein to the respiratory tract of a subject, at a nominal dose of about 500 μg/day for a period of seven days or longer, may result in a systemic Cₘₐₓ of Compound A at Day 7 of about 300-350 pg/mL, e.g., about 310 μg/mL, about 320 μg/mL, or about 330 μg/mL. Administering a dry powder disclosed herein to the respiratory tract of a subject, at a nominal dose of about 500 μg/day for a period of fourteen days or longer, may result in a systemic Cₘₐₓ of Compound A at Day 14 of about 320-370 pg/mL, e.g., about 340 μg/mL, about 350 μg/mL, or about 360 μg/mL.

Administering a dry powder disclosed herein to the respiratory tract of a subject, at a nominal dose of about 500 μg, may result in a systemic AUC_{0-24 h} of Compound A of about 1700-2200 pg*h/mL, e.g., about 1900 pg*h/mL, about 2000 pg*h/mL, or about 2100 pg*h/mL. Administering a dry powder disclosed herein to the respiratory tract of a subject, at a nominal dose of about 500 μg/day for a period of seven days or longer, may result in a systemic AUC_{0-24 h} of Compound A at Day 7 of about 3400-3800 pg*h/mL, e.g., about 3500 pg*h/mL, about 3600 pg*h/mL, or about 3700 pg*h/mL. Administering a dry powder disclosed herein to the respiratory tract of a subject, at a nominal dose of about 500 μg/day for a period of fourteen days or longer, may result in a systemic AUC_{0-24 h} of Compound A at Day 14 of about 3800-4100 pg*h/mL, e.g., about 3900 pg*h/mL, about 4000 pg*h/mL, or about 4100 pg*h/mL.

Administering a dry powder disclosed herein to the respiratory tract of a subject, at a nominal dose of about 500 μg/day for a period of seven days or longer (e.g., 2 weeks, 3 weeks, 4 weeks, 2 months, 3 months, 6 months, or longer) may result in accumulation of Compound A (based on AUC_{0-24 h}) of less than 2.1.

Administering a dry powder disclosed herein to the respiratory tract of a subject may achieve Tₘₐₓ of Compound A in 0.75 hours or less, post dose.

### EXEMPLIFICATION

Materials used in the following Examples and their sources are listed below. Sodium chloride, sodium sulfate, polysorbate 80, ammonium hydroxide, mannitol, magnesium stearate, and L-leucine were obtained from Sigma-Aldrich Co. (St. Louis, MO), Spectrum Chemicals (Gardena, CA), Applichem (Maryland Heights, MO), Alfa Aesar (Tewksbury, MA), Thermo Fisher (Waltham, MA), Croda Chemicals (East Yorkshire, United Kingdom) or Merck (Darmstadt, Germany). Ultrapure (Type II ASTM) water was from a water purification system (Millipore Corp., Billerica, MA), or equivalent.

### Methods:

Geometric of Volume Diameter of Suspensions. Volume median diameter (x50 or Dv50), which may also be referred to as volume median geometric diameter (VMGD), of the active agent suspensions was determined using a laser diffraction technique. The equipment consisted of a Horiba LA-950 instrument outfitted with an automated recirculation system for sample handling and removal or a fixed-volume sample cuvette. The sample to a dispersion media, consisting of either deionized water or deionized water with less than 0.5% of a surfactant such as polysorbate 80 or sodium dodecyl sulfate. Ultrasonic energy can be applied to aid in dispersion of the suspension. When the laser transmission was in the correct range, the sample was sonicated for 60 seconds at a setting of 5. The sample was then measured and the particle size distribution reported.

Geometric or Volume Diameter of Dry Powders. Volume median diameter (x50 or Dv50), which may also be referred to as volume median geometric diameter (VMGD), of the dry powder formulations was determined using a laser diffraction technique. The equipment consisted of a HELOS diffractometer and a RODOS dry powder disperser (Sympatec, Inc., Princeton, NJ). The RODOS disperser applies a shear force to a sample of particles, controlled by the regulator pressure (typically set at 1.0 bar with maximum orifice ring pressure) of the incoming compressed dry air. The pressure settings may be varied to vary the amount of energy used to disperse the powder. For example, the dispersion energy may be modulated by changing the regulator pressure from 0.2 bar to 4.0 bar. Powder sample is dispensed from a microspatula into the RODOS funnel. The dispersed particles travel through a laser beam where the resulting diffracted light pattern produced is collected, typically using an R1 lens, by a series of detectors. The ensemble diffraction pattern is then translated into a volume-based particle size distribution using the Fraunhofer diffraction model, on the basis that smaller particles diffract light at larger angles. Using this method, the span of the distribution was also determined per the formula (Dv[90]−Dv[10)/Dv[50]. The span value gives a relative indication of the polydispersity of the particle size distribution.

Aerodynamic Performance via Andersen Cascade Impactor. The aerodynamic properties of the powders dispersed from an inhaler device were assessed with an Mk-II 1 ACFM Andersen Cascade Impactor (Copley Scientific Limited, Nottingham, UK) (ACI). The ACI instrument was run in controlled environmental conditions of 18 to 25° C. and relative humidity (RH) between 25 and 35%. The instrument consists of eight stages that separate aerosol particles based on inertial impaction. At each stage, the aerosol stream passes through a set of nozzles and impinges on a corresponding impaction plate. Particles having small enough inertia will continue with the aerosol stream to the next stage, while the remaining particles will impact upon the plate. At each successive stage, the aerosol passes through nozzles at a higher velocity and aerodynamically smaller particles are collected on the plate. After the aerosol passes through the final stage, a filter collects the smallest particles that remain, called the “final collection filter”. Gravimetric and/or chemical analyses can then be performed to determine the particle size distribution. A short stack cascade impactor, also referred to as a collapsed cascade impactor, is also utilized to allow for reduced labor time to evaluate two aerodynamic particle size cut-points. With this collapsed cascade impactor, stages are eliminated except those required to establish fine and coarse particle fractions. The impaction techniques utilized allowed for the collection of two or eight separate powder fractions. The capsules (HPMC, Size 3; Capsugel Vcaps, Peapack, NJ) were filled with powder to a specific weight and placed in a hand-held, breath-activated dry powder inhaler (DPI) device, the high resistance RS01 DPI or the ultra-high resistance UHR2 DPI (both by Plastiape, Osnago, Italy). The capsule was punctured and the powder was drawn through the cascade impactor operated at a flow rate of 60.0 L/min for 2.0 s. At this flowrate, the calibrated cut-off diameters for the eight stages are 8.6, 6.5, 4.4, 3.3, 2.0, 1.1, 0.5 and 0.3 microns and for the two stages used with the short stack cascade impactor, based on the Andersen Cascade Impactor, the cut-off diameters are 5.6 microns and 3.4 microns. The fractions were collected by placing filters in the apparatus and determining the amount of powder that impinged on them by gravimetric measurements or chemical measurements on an HPLC.

Aerodynamic Performance via Next Generation Impactor. The aerodynamic properties of the powders dispersed from an inhaler device were assessed with a Next Generation Impactor (Copley Scientific Limited, Nottingham, UK) (NGI). For measurements utilizing the NGI, the NGI instrument was run in controlled environmental conditions of 18 to 25° C. and relative humidity (RH) between 25 and 35%. The instrument consists of seven stages that separate aerosol particles based on inertial impaction and can be operated at a variety of air flow rates. At each stage, the aerosol stream passes through a set of nozzles and impinges on a corresponding impaction surface. Particles having small enough inertia will continue with the aerosol stream to the next stage, while the remaining particles will impact upon the surface. At each successive stage, the aerosol passes through nozzles at a higher velocity and aerodynamically smaller particles are collected on the plate. After the aerosol passes through the final stage, a micro-orifice collector collects the smallest particles that remain. Gravimetric and/or chemical analyses can then be performed to determine the particle size distribution. The capsules (HPMC, Size 3; Capsugel Vcaps, Peapack, NJ) were filled with powder to a specific weight and placed in a hand-held, breath-activated dry powder inhaler (DPI) device, the high resistance RS01 DPI or the ultra-high resistance RS01 DPI (both by Plastiape, Osnago, Italy). The capsule was punctured and the powder was drawn through the cascade impactor operated at a specified flow rate for 2.0 Liters of inhaled air. At the specified flow rate, the cut-off diameters for the stages were calculated. The fractions were collected by placing wetted filters in the apparatus and determining the amount of powder that impinged on them by chemical measurements on an HPLC.

Fine Particle Dose. The fine particle dose indicates the mass of one or more therapeutics in a specific size range and can be used to predict the mass which will reach a certain region in the respiratory tract. The fine particle dose can be measured gravimetrically or chemically via either an ACI or NGI. If measured gravimetrically, since the dry particles are assumed to be homogenous, the mass of the powder on each stage and collection filter can be multiplied by the fraction of therapeutic agent in the formulation to determine the mass of therapeutic. If measured chemically, the powder from each stage or filter is collected, separated, and assayed for example on an HPLC to determine the content of the therapeutic. The cumulative mass deposited on each of the stages at the specified flow rate is calculated and the cumulative mass corresponding to a 5.0 micrometer diameter particle is interpolated. This cumulative mass for a single dose of powder, contained in one or more capsules, actuated into the impactor is equal to the fine particle dose less than 5.0 microns (FPD <5.0 microns).

Mass Median Aerodynamic Diameter. Mass median aerodynamic diameter (MMAD) was determined using the information obtained by the Andersen Cascade Impactor (ACI). The cumulative mass under the stage cut-off diameter is calculated for each stage and normalized by the recovered dose of powder. The MMAD of the powder is then calculated by linear interpolation of the stage cut-off diameters that bracket the 50th percentile. An alternative method of measuring the MMAD is with the Next Generation Impactor (NGI). Like the ACI, the MMAD is calculated with the cumulative mass under the stage cut-off diameter is calculated for each stage and normalized by the recovered dose of powder. The MMAD of the powder is then calculated by linear interpolation of the stage cut-off diameters that bracket the 50th percentile.

Emitted Geometric or Volume Diameter. The volume median diameter (Dv50) of the powder after it is emitted from a dry powder inhaler, which may also be referred to as volume median geometric diameter (VMGD), was determined using a laser diffraction technique via the Spraytec diffractometer (Malvern, Inc.). Powder was filled into size 3 capsules (V-Caps, Capsugel) and placed in a capsule based dry powder inhaler (RS01 Model 7 High resistance, Plastiape, Italy), or DPI, and the DPI sealed inside a cylinder. The cylinder was connected to a positive pressure air source with steady air flow through the system measured with a mass flow meter and its duration controlled with a timer controlled solenoid valve. The exit of the dry powder inhaler was exposed to room pressure and the resulting aerosol jet passed through the laser of the diffraction particle sizer (Spraytec) in its open bench configuration before being captured by a vacuum extractor. The steady air flow rate through the system was initiated using the solenoid valve. A steady air flow rate was drawn through the DPI typically at 60 L/min for a set duration, typically of 2 seconds. Alternatively, the air flow rate drawn through the DPI was sometimes run at 15 L/min, 20 L/min, or 30 L/min. The resulting geometric particle size distribution of the aerosol was calculated from the software based on the measured scatter pattern on the photodetectors with samples typically taken at 1000 Hz for the duration of the inhalation. The Dv50, GSD, FPF<5.0 μm measured were then averaged over the duration of the inhalation.

Emitted Dose (ED) refers to the mass of therapeutic which exits a suitable inhaler device after a firing or dispersion event. The ED is determined using a method based on USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, MD, 13^{th} Revision, 222-225, 2007. Contents of capsules are dispersed using either the RS01 HR inhaler at a pressure drop of 4 kPa and a typical flow rate of 60 LPM or the UHR2 RS01 at a pressure drop of 4 kPa and a typical flow rate of 39 LPM. The emitted powder is collected on a filter in a filter holder sampling apparatus. The sampling apparatus is rinsed with a suitable solvent such as water and analyzed using an HPLC method. For gravimetric analysis a shorter length filter holder sampling apparatus is used to reduce deposition in the apparatus and the filter is weighed before and after to determine the mass of powder delivered from the DPI to the filter. The emitted dose of therapeutic is then calculated based on the content of therapeutic in the delivered powder. Emitted dose can be reported as the mass of therapeutic delivered from the DPI or as a percentage of the filled dose. ED can also be calculated from the results generated by Next Generation Impactor (NGI) experiments, through summation of all of the drug or powder assayed from the mouthpiece adapter, NGI induction port, and all of the stages within the NGI. The results generated through ED testing per USP 601 and the results generated via the NGI are typically in good agreement.

Thermogravimetric Analysis: Thermogravimetric analysis (TGA) was performed using either the Q500 model or the Discovery model thermogravimetric analyzer (TA Instruments, New Castle, DE). The samples were either placed into an open aluminum DSC pan or a sealed aluminum DSC pan that was then automatically punched open prior to the time of test. Tare weights were previously recorded by the instrument. The following method was employed: Ramp 5.00° C./min from ambient (˜35° C.) to 200° C. The weight loss was reported as a function of temperature up to 140° C. TGA allows for the calculation of the content of volatile compounds within the dry powder. When utilizing processes with water alone, or water in conjunction with volatile solvents, the weight loss via TGA is a good estimate of water content.

X-Ray Powder Diffraction: The crystalline character of the formulations was assessed via powder X-ray diffraction (PXRD). A 20-30 mg sample of material is analyzed in a powder X-ray diffractometer (D8 Discover with LINXEYE detector; Bruker Corporation, Billerica, MA or equivalent) using a Cu X-ray tube with 1.5418 A at a data accumulation time 1.2 second/step over a scan range of 5 to 45°2θ and a step size of 0.02°2θ.

Compound A Content/Purity using HPLC. A high performance liquid chromatography (HPLC) method utilizing a reverse phase C18 column coupled to an ultraviolet (UV) detector has been developed for the identification, bulk content, assay, CUPMD and impurities analysis of formulations of Compound A. The reverse phase column is equilibrated to 40° C. and the autosampler is set to 5° C. The mobile phases, 10 mM ammonium acetate with 0.1% TFA (mobile phase A) and acetonitrile (mobile phase B) are used in a gradient elution from a ratio of 70:30 (A:B) to 0:100 (A:B), over the course of a 20 minute run time. Detection is by UV at 260 nm and the injection volume is 30 μL. Compound A content in powders are quantified relative to a standard curve. Formula strength can be determined by measuring the amount of Compound A in the bulk powder with HPLC, then dividing the measured amount by the nominal amount of Compound A in the powder. A formula strength of a dry powder disclosed herein may be between about 90% and about 105%, e.g., about 93%, about 94%, about 95%, about 96%, about 97%, about 98%, about 99%, about 100%, about 101%, or about 102%.

Particle Size Reduction. The particle size distribution of the crystalline active agent can be modulated using a number of techniques familiar to those of skill in the art, including but not limited to, high-pressure homogenization, high-shear homogenization, jet-milling, pin milling, microfluidization, or wet milling (also known as ball milling, pearl milling or bead milling). Wet milling is often preferred, as it is able to achieve a wide range of particle size distributions, including those in the nanometer (<1 μm) size domain.

Particle Size Reduction using Low Energy Wet Milling. One technique for reducing the particle size of the active agent was via low energy wet milling, (also known as roller milling, or jar milling). Suspensions of the active agent were prepared in an anti-solvent, which can be water, or any solvent in which the active agent is not appreciably soluble. Stabilizers, which can be, but are not limited to, non-ionic surfactants or amphiphilic polymers, are then added to the suspension along with milling media, which can be, but are not limited to, spherical with high wear resistance and in the size range from 0.03 to 0.70 millimeters in diameter. The vessels containing the suspensions are then rotated using ajar mill (US Stoneware, East Palestine, OH USA) while taking samples periodically to assess particle size (LA-950, HORIBA, Kyoto, Japan). When the particle size is sufficiently reduced, or when a particle size minimum is reached, the suspension is strained through a sieve to remove the milling media, and the product recovered.

Particle Size Reduction using High Energy Wet Milling. Another technique for reducing the particle size of the active agent was via high-energy wet milling using a rotor-stator, or agitated media mill. Suspensions of the active agent were prepared in an anti-solvent, which can be water, or any solvent in which the active agent is not appreciably soluble. Stabilizers, which can be, but are not limited to, non-ionic surfactants or amphiphilic polymers, are then added to the suspension along with milling media, which can be, but are not limited to, spherical with high wear resistance and in the size range from 0.03 to 0.70 millimeters in diameter. The suspensions are then charged into the mill, which can be operated in either batch or recirculation mode. The process consists of the suspension and milling media being agitated within the milling chamber, which increases the energy input to the system and accelerates the particle size reduction process. The milling chamber and recirculation vessel are jacketed and actively cooled to avoid temperature increases in the product. The agitation rate and recirculation rate of the suspension are controlled during the process. Samples are taken periodically to assess particle size (LA-950, HORIBA, Kyoto, Japan). When the particle size is sufficiently reduced, or when a particle size minimum is reached, the suspension is discharged from the mill.

Particle Size Reduction using Microfluidization. Another technique for reducing the particle size distribution of the active agent was via Microfluidization. Microfluidizer-based processing is a high-shear wet-processing unit operation utilized for particle size reduction of liquids and solids. The unit can be configured with various interaction chambers, which are cylindrical modules with specific orifice and channel designs through which fluid is passed at high pressures to control shear rates. Product enters the unit via the inlet reservoir and is forced into the fixed-geometry interaction chamber at speeds up to 400 m/sec by a high-pressure pump. It is then effectively cooled, if required, and collected in the output reservoir. The process can be repeated as necessary (e.g. multiple “passes”) to achieve the particle size targets. Particle size of the active agent is monitored periodically via laser diffraction (LA-950, HORIBA, Kyoto, Japan). When the particle size is sufficiently reduced, or when a particle size minimum is reached, the suspension is recovered from the unit.

Particle Size Reduction using Jet Milling. Another technique for reducing the particle size distribution of the active agent was via jet milling. Jet mills utilize fluid energy (compressed air or gas) to grind and classify, in a single chamber with no moving parts. Activated by high pressure air, the particles are accelerated into a high speed rotation in a shallow grinding chamber. As the particles impact on one another their size is reduced. Centrifugal force holds larger particles in the grinding rotation area until they have achieved the desired fine particle size. Centripetal force drags the desired particles towards the static classifier where they are allowed to exit upon achieving the correct particle size. The final particle size is controlled by varying the rate of the feed and propellant pressure.

Liquid Feedstock Preparation for Spray Drying. Spray drying homogenous particles requires that the ingredients of interest be solubilized in solution or suspended in a uniform and stable suspension. The feedstock can utilize water, or a combination of water and other miscible solvents such as alcohols or ketones, as the solvent in the case of solutions, or as the continuous phase in the case of suspensions. Feedstocks of the various formulations were prepared by dissolving the soluble components in the desired solvent(s) followed by dispersing the surfactant-stabilized active agent-containing suspension in the resulting solution while mixing, although the process is not limited to this specific order of operations.

Spray Drying Using Niro Spray Dryer. Dry powders were produced by spray drying utilizing a Niro Mobile Minor spray dryer (GEA Process Engineering Inc., Columbia, MD) with powder collection from a cyclone, a product filter or both. Atomization of the liquid feed was performed using a co-current two-fluid nozzle either from Niro (GEA Process Engineering Inc., Columbia, MD) or a Spraying Systems (Carol Stream, IL) 1/4 J two-fluid nozzle with gas cap 67147 and fluid cap 2850SS, although other two-fluid nozzle setups are also possible. In some embodiments, the two-fluid nozzle can be in an internal mixing setup or an external mixing setup. Additional atomization techniques include rotary atomization or a pressure nozzle. The liquid feed was fed using gear pumps (Cole-Parmer Instrument Company, Vernon Hills, IL) directly into the two-fluid nozzle or into a static mixer (Charles Ross & Son Company, Hauppauge, NY) immediately before introduction into the two-fluid nozzle. An additional liquid feed technique includes feeding from a pressurized vessel. Nitrogen or air may be used as the drying gas, provided that moisture in the air is at least partially removed before its use. Pressurized nitrogen or air can be used as the atomization gas feed to the two-fluid nozzle. The drying gas inlet temperature can range from 70° C. to 300° C. and outlet temperature from 30° C. to 120° C. with a liquid feedstock rate of 10 mL/min to 100 mL/min. The gas supplying the two-fluid atomizer can vary depending on nozzle selection and for the Niro co-current two-fluid nozzle can range from 5 kg/hr to 50 kg/hr or for the Spraying Systems 1/4 J two-fluid nozzle can range from 30 g/min to 150 g/min. The atomization gas rate can be set to achieve a certain gas to liquid mass ratio, which directly affects the droplet size created. The pressure inside the drying drum can range from +3 “WC to −6 “WC. Spray dried powders can be collected in a container at the outlet of the cyclone, onto a cartridge or baghouse filter, or from both a cyclone and a cartridge or baghouse filter.

Spray Drying Using Büchi Spray Dryer. Dry powders were prepared by spray drying on a Büchi B-290 Mini Spray Dryer (BÜCHI Labortechnik AG, Flawil, Switzerland) with powder collection from either a standard or High Performance cyclone. The system was run either with air or nitrogen as the drying and atomization gas in open-loop (single pass) mode. When run using air, the system used the Büchi B-296 dehumidifier to ensure stable temperature and humidity of the air used to spray dry. Furthermore, when the relative humidity in the room exceeded 30% RH, an external LG dehumidifier (model 49007903, LG Electronics, Englewood Cliffs, NJ) was run constantly. When run using nitrogen, a pressurized source of nitrogen was used. Furthermore, the aspirator of the system was adjusted to maintain the system pressure at −2.0″ water column. Atomization of the liquid feed utilized a Büchi two-fluid nozzle with a 1.5 mm diameter or a Schlick 970-0 atomizer with a 0.5 mm liquid insert (Düsen-Schlick GmbH, Coburg, Germany). Inlet temperature of the process gas can range from 100° C. to 220° C. and outlet temperature from 30° C. to 120° C. with a liquid feedstock flowrate of 3 mL/min to 10 mL/min. The two-fluid atomizing gas ranges from 25 mm to 45 mm (300 LPH to 530 LPH) for the Büchi two-fluid nozzle and for the Schlick atomizer an atomizing air pressure of upwards of 0.3 bar. The aspirator rate ranges from 50% to 100%.

Stability Assessment: The physicochemical stability and aerosol performance of select formulations were assessed at 2-8° C., 25° C./60% RH, and when material quantities permitted, 40° C./75% RH as detailed in the International Conference on Harmonization (ICH) Q1 guidance. Stability samples were stored in calibrated chambers (Darwin Chambers Company Models PH024 and PH074, St. Louis. MO). Bulk powder samples were weighed into amber glass vials, sealed under 30% RH, and induction-sealed in aluminum pouches (Drishield 3000, 3M, St. Paul, MN) with silica desiccant (2.0 g, Multisorb Technologies, Buffalo, NY). Additionally, to assess the stability of the formulations in capsules, the target mass of powder was weighed by hand into a size 3, HPMC capsule (Capsugel Vcaps, Peapack, NJ) at 30% RH or less. Filled capsules were then aliquoted into high-density polyethylene (HDPE) bottles and induction sealed in aluminum pouches with silica desiccant.

### Example 1

Compatibility Studies of Compound A with Excipients

### A. Sample Preparation.

The nanocrystalline Compound A was prepared by compounding 2.00 g of Compound A (Janssen, Lot A13CD0665) and 0.21 g of polysorbate 80 (Sigma, Lot BCB59994) in 17.80 g of water in ajar and stirring until Compound A was fully suspended. 37.3 g of 200 μm yttria-stabilized zirconia (YTZ) milling media (Tosoh Corp, Tokyo Japan) was then added to the suspension. The jar containing the suspension was milled at 200 RPM for 18 hours before being collected. The final median particle size (D-v(50)) of the milled suspension was 136 nm.

Feedstock solutions were prepared and used to manufacture Formulations I-VI, dry powders composed of Compound A, polysorbate 80 and one excipient for use in excipient compatibility studies. Drug loads of 5 wt % and 50 wt % Compound A on a dry basis, were targeted.

The feedstock solutions that were used to spray dry the dry powders for Formulations I-V were made as follows. The required quantity of water was weighed into a suitably sized glass vessel. The excipient was added to the water and the solution allowed to stir until visually clear. The Compound A-containing suspension was then added to the excipient solution and stirred until visually homogenous. The feedstocks were then spray-dried. Feedstock masses ranged from 51.5 g to 100 g, which supported manufacturing campaigns from 10 to 20 minutes. The feedstock to support Formulation I was formulated at 15 g/L solids concentration while Formulations II-V were formulated at 30 g/L solids concentration. Table 2 lists the components of the feedstocks used in preparation of the excipient compatibility samples.

The feedstock solution that was used to spray dry Formulation VI was manufactured using micronized Compound A in place of nanocrystalline Compound A as follows. The required quantity of water was weighed into a suitably sized glass vessel. The excipient was added to the water and the solution allowed to stir until visually clear. The polysorbate 80 was added to the solution and allowed to stir. The Compound A was then weighed, added to the excipient solution and stirred until visually homogenous. The feedstock was formulated at 30 g/L solids concentration. The feedstock compositions for the formulations are included in Table 2.

TABLE 2
Formulation I-VI excipient compatibility feedstock compositions

| | Excipient | Water | Compound | Polysorbate | Excipient | Total mass |
|---|---|---|---|---|---|---|
| Formulation | Name | (g) | A (g) | 80 (g) | (g) | (g) |
| I | Leucine | 98.465 | 0.075 | 0.008 | 1.421 | 99.969 |
| II | Mannitol | 50.021 | 0.075 | 0.008 | 1.421 | 51.525 |
| III | Sodium | 50.006 | 0.075 | 0.008 | 1.416 | 51.505 |
| | chloride | | | | | |
| IV | Sodium | 50.005 | 0.075 | 0.008 | 1.417 | 51.505 |
| | sulfate | | | | | |
| V | Trehalose | 50.004 | 0.075 | 0.008 | 1.558 | 51.645 |
| VI | Sodium | 39.973 | 0.600 | 0.061 | 0.541 | 41.175 |
| | chloride | | | | | |

Dry powders were manufactured from these feedstocks by spray drying on the B3chi B-290 Mini Spray Dryer (BÜCHI Labortechnik AG, Flawil, Switzerland) with cyclone powder collection. The system was run in open-loop (single pass) mode using nitrogen as the drying and atomization gas. The aspirator of the system was adjusted to maintain the system pressure at −2.5″ to −3.0″ water column.

The following spray drying conditions were followed to manufacture the dry powders. The process gas inlet temperature was 128° C. to 145° C., the process gas outlet temperature was 60° C., the drying gas flowrate was 17 kg/hr, the atomization gas flowrate was 17.5 g/min, the atomization gas backpressure at the atomizer inlet was 80 psig and the liquid feedstock flowrate was 4 mL/min. The resulting dry powder formulations are reported in Table 3.

TABLE 3
Formulation I-VI excipient compatibility
dry powder compositions, dry basis

| Compati- | |
|---|---|
| bility | |
| Powder | Dry Powder Composition (w/w), dry basis |
| I | 5.0% Compound A, 0.5% polysorbate 80, 94.5% leucine |
| II | 5.0% Compound A, 0.5% polysorbate 80, 94.5% mannitol |
| III | 5.0% Compound A, 0.5% polysorbate 80, 94.5% sodium |
| | chloride |
| IV | 5.0% Compound A, 0.5% polysorbate 80, 94.5% sodium |
| | sulfate |
| V | 5.0% Compound A, 0.5% polysorbate 80, 94.5% trehalose |
| VI | 50.0% Compound A, 5.0% polysorbate 80, 45.0% sodium |
| | chloride |

### B. Study Design

The spray dried Compound A formulations were exposed to stressed storage conditions for up to two weeks to assess the potential for excipient-induced degradation. The powders were added to 4 mL vials and exposed to 60° C./30% RH, 70° C./10% RH and 80° C./50% RH. Testing for impurities occurred at t=0, t=3 days, t=7 days and t=14 days for samples held at 60° C./30% RH and 70° C./10% RH and at t=0, t=3 days and t=7 days for samples held at 80° C./50% RH. Control samples including Formulation VI, comprising 50% micronized Compound A, 45% sodium chloride, and 5% polysorbate 80 as well as as-received micronized Compound A were included in an attempt to decouple particle size effects and/or the degradation potential of polysorbate 80.

### C. Study Results

The excipient compatibility results are found in Table 4. At 60° C./30% RH and 70° C./10% RH, impurity levels in Formulations II-VI were similar to one another and to those observed for the micronized Compound A control. At 80° C./50% RH, impurity levels after 7 days for Formulations II-VI were similar to one another, but about 5-fold those observed for the micronized Compound A control under the same condition. Formulation I saw a greater increase in impurity growth than all other samples, at times reaching about 15-fold the impurity level of the micronized Compound A control.

TABLE 4
Formulation I-VI excipient compatibility results

| | | | | | Total | Total | |
|---|---|---|---|---|---|---|---|
| | | | Time | | Known | Unknown | Total |
| | Compound | Excipient | Point | | Impurities | Impurities | Impurities |
| Formulation | A (wt. %) | (wt. %) | (days) | Condition | (% w/w) | (% w/w) | (% w/w) |
| n/a | 100 | n/a | 0 | n/a | 0.22 | 0.07 | 0.29 |
| | | | 3 | 60 C./30% | 0.20 | 0.08 | 0.28 |
| | | | 7 | RH | 0.17 | 0.09 | 0.26 |
| | | | 14 | | 0.14 | 0.07 | 0.21 |
| | | | 3 | 70 C./10% | 0.19 | 0.11 | 0.29 |
| | | | 7 | RH | 0.17 | 0.14 | 0.31 |
| | | | 14 | | 0.13 | 0.08 | 0.20 |
| | | | 3 | 80 C./50% | 0.16 | 0.09 | 0.24 |
| | | | 7 | RH | 0.14 | 0.18 | 0.32 |
| VI | 50 | Sodium | 0 | n/a | 0.22 | 0.03 | 0.25 |
| | | chloride | 3 | 60 C./30% | 0.23 | 0.06 | 0.29 |
| | | (45%) | 7 | RH | 0.23 | 0.03 | 0.26 |
| | | | 14 | | 0.30 | 0.04 | 0.34 |
| | | | 3 | 70 C./10% | 0.23 | 0.11 | 0.34 |
| | | | 7 | RH | 0.25 | 0.03 | 0.28 |
| | | | 14 | | 0.32 | 0.04 | 0.36 |
| | | | 3 | 80 C./50% | 0.62 | 0.04 | 0.66 |
| | | | 7 | RH | 0.83 | 0.20 | 1.03 |
| I | 5 | Leucine | 0 | n/a | 0.22 | 0.11 | 0.32 |
| | | (94.5%) | 3 | 60 C./30% | 0.09 | 0.85 | 0.93 |
| | | | 7 | RH | 0.13 | 1.21 | 1.34 |
| | | | 14 | | 0.29 | 0.38 | 1.00 |
| | | | 3 | 70 C./10% | 0.07 | 1.41 | 1.48 |
| | | | 7 | RH | 0.18 | 2.26 | 2.44 |
| | | | 14 | | 0.44 | 0.76 | 1.92 |
| | | | 3 | 80 C./50% | 0.28 | 2.83 | 3.11 |
| | | | 7 | RH | 0.27 | 4.56 | 4.83 |
| IV | 5 | Sodium | 0 | n/a | 0.20 | 0.06 | 0.26 |
| | | sulfate | 3 | 60 C./30% | 0.10 | 0.03 | 0.13 |
| | | (94.5%) | 7 | RH | 0.18 | 0.06 | 0.23 |
| | | | 14 | | 0.24 | 0.07 | 0.34 |
| | | | 3 | 70 C./10% | 0.23 | 0.14 | 0.37 |
| | | | 7 | RH | 0.32 | 0.07 | 0.39 |
| | | | 14 | | 0.41 | 0.08 | 0.53 |
| | | | 3 | 80 C./50% | 0.58 | 0.45 | 1.02 |
| | | | 7 | RH | 0.63 | 1.00 | 1.62 |
| III | 5 | Sodium | 0 | n/a | 0.20 | 0.10 | 0.30 |
| | | chloride | 3 | 60 C./30% | 0.24 | 0.13 | 0.37 |
| | | (94.5%) | 7 | RH | 0.21 | 0.04 | 0.25 |
| | | | 14 | | 0.27 | 0.03 | 0.29 |
| | | | 3 | 70 C./10% | 0.26 | 0.14 | 0.40 |
| | | | 7 | RH | 0.34 | 0.04 | 0.38 |
| | | | 14 | | 0.43 | 0.07 | 0.51 |
| | | | 3 | 80 C./50% | 0.64 | 0.32 | 0.95 |
| | | | 7 | RH | 0.66 | 0.90 | 1.56 |
| V | 5 | Trehalose | 0 | n/a | 0.20 | 0.10 | 0.30 |
| | | (94.5%) | 3 | 60 C./30% | 0.23 | 0.08 | 0.31 |
| | | | 7 | RH | 0.23 | 0.03 | 0.26 |
| | | | 14 | | 0.22 | 0.05 | 0.28 |
| | | | 3 | 70 C./10% | 0.19 | 0.08 | 0.27 |
| | | | 7 | RH | 0.23 | 0.00 | 0.23 |
| | | | 14 | | 0.26 | 0.04 | 0.29 |
| | | | 3 | 80 C./50% | 0.33 | 0.09 | 0.42 |
| | | | 7 | RH | 0.98 | 0.48 | 1.46 |
| II | 5 | Mannitol | 0 | n/a | 0.19 | 0.08 | 0.27 |
| | | (94.5%) | 3 | 60 C./30% | 0.18 | 0.09 | 0.27 |
| | | | 7 | RH | 0.16 | 0.11 | 0.27 |
| | | | 14 | | 0.30 | 0.05 | 0.36 |
| | | | 3 | 70 C./10% | 0.18 | 0.14 | 0.32 |
| | | | 7 | RH | 0.21 | 0.08 | 0.29 |
| | | | 14 | | 0.31 | 0.51 | 0.84 |
| | | | 3 | 80 C./50% | 0.43 | 0.32 | 0.75 |
| | | | 7 | RH | 0.63 | 1.04 | 1.67 |

### Example 2. Dry Powder Formulations of Nanocrystalline Compound a Containing Sodium Sulfate/Mannitol

### A. Powder Preparation.

The nanocrystalline Compound A used to support Formulation VII was prepared by compounding 25.3 g of Compound A (Janssen, Lot I15ED2302) in 222.5 g of water and 2.5 g of polysorbate 80 (Sigma, Lot BCBR8595V) in ajar and stirring until the Compound A was fully suspended. 487.6 g of 200 μm YTZ milling media (Tosoh Corp, Tokyo Japan) was added and the suspension was milled at 150 RPM for 34 hours before being collected. The final median particle size (Dv(50)) of the milled suspension was 127 nm.

The nanocrystalline Compound A used to support Formulation VIII was prepared as follows. Four individual suspensions were formulated for milling. In each jar, 2.0±0.05 g of Compound A (Janssen, Lot I15ED2302) was compounded with 0.20±0.005 g of polysorbate 80 (Sigma, Lot BCB5994) in 17.8±0.1 g of water and stirred until the Compound A was fully suspended. 35±5 g of 200 μm YTZ milling media (Tosoh Corp, Tokyo Japan) was added to each suspension. The suspensions were milled at 200 RPM for 21-22 hours before being collected. The four suspensions were pooled together after discharging the media. The final median particle size (Dv(50)) of the milled suspension was 146 nm.

Feedstock solutions were prepared and used to manufacture dry powders composed of nanocrystalline Compound A, polysorbate 80 and additional excipients. Drug loads of 2 wt % and 10 wt % Compound A, on a dry basis, were targeted. The feedstock solutions that were used to spray dry powders were made as follows. The required quantity of water was weighed into a suitably sized glass vessel. The excipients were added to the water and the solution allowed to stir until visually clear. The Compound A-containing suspension was then added to the excipient solution and stirred until visually homogenous. The feedstocks were then spray-dried. Feedstock masses ranged from 97 g to 980 g, which supported manufacturing campaigns from 23 to 235 minutes. Table 5 lists the components of the feedstocks used in preparation of the dry powders.

TABLE 5
Formulation VII-VIII feedstock compositions

| | | | | Sodium | | |
|---|---|---|---|---|---|---|
| Formu- | Water | Compound | PS80 | sulfate | Mannitol | Total |
| lation | (g) | A (g) | (g) | (g) | (g) | mass (g) |
| VII | 97.004 | 0.060 | 0.006 | 1.466 | 1.467 | 100.003 |
| VIII | 980.0 | 2.0 | 0.2 | 8.9 | 8.9 | 1000.0 |

PS80 = Polysorbate 80.

Dry powders of Formulations VII and VIII were manufactured from these feedstocks by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. The aspirator of the system was adjusted to maintain the system pressure at −2.0 to −3.0″ water column.

The following spray drying conditions were followed to manufacture the dry powders. For Formulation VII, the liquid feedstock solids concentration was 30 g/kg, the process gas inlet temperature was 133° C. to 136° C., the process gas outlet temperature was 65° C., the drying gas flowrate was 17 kg/hr, the atomization gas flowrate was 19.5 g/min, the atomization gas backpressure at the atomizer inlet was 80 psig and the liquid feedstock flowrate was 4 mL/min. For Formulation VIII, the liquid feedstock solids concentration was 20 g/kg, the process gas inlet temperature was 139° C. to 156° C., the process gas outlet temperature was 65° C., the drying gas flowrate was 16.9-17.1 kg/hr, the atomization gas flowrate was 19.5-19.7 g/min, the atomization gas backpressure at the atomizer inlet was 80 psig and the liquid feedstock flowrate was 4 mL/min. The resulting dry powder formulations are reported in Table 6.

TABLE 6
Formulation VII-VIII dry powder compositions, dry basis

| Formulation | Dry Powder Composition (w/w), dry basis |
|---|---|
| VII | 2.0% Compound A, 0.2% PS80, 48.9% sodium sulfate, |
| | 48.9% mannitol |
| VIII | 10.0% Compound A, 1% polysorbate 80, 44.5% sodium |
| | sulfate, 44.5% mannitol |

### B. Powder Characterization.

The bulk particle size characteristic for Formulation VIII is found in Table 7. The span at 1 bar of 1.72 for Formulation VIII indicates a relatively narrow size distribution. The 1 bar/4 bar dispersibility ratio 1.05 for Formulation VIII indicates the size of the powder is relatively independent of dispersion energy, a desirable characteristic which allows similar particle dispersion across a range of dispersion energies.

TABLE 7
Formulation VIII bulk particle size

| | 0.5 bar | | 1 bar | | 4 bar | | |
|---|---|---|---|---|---|---|---|
| Formu- | Dv[50] | | Dv[50] | | Dv[50] | | 1 bar:4 bar |
| lation | (μm) | Span | (μm) | Span | (μm) | Span | Dv[50] ratio |
| VIII | 2.31 | 1.63 | 2.08 | 1.72 | 1.97 | 1.81 | 1.05 |

The aerodynamic particle size, fine particle fractions and fine particle doses of Formulations VII and VIII measured and/or calculated with a Next Generation Impactor (NGI) are reported in Table 8. The fine particle dose for Formulations VII and VIII indicate a high percentage of the nominal dose which is filled into the capsule reaches the impactor stages (50.5% and 42.6%, respectively) and so would be predicted to be delivered to the lungs. The MMAD of Formulations VII and VIII were 3.81 μm and 3.35 μm respectively, indicating deposition in the central and conducting airways.

TABLE 8
Formulation VII-VIII aerodynamic particle size

| | MMAD | FPD < 5 μm |
|---|---|---|
| Formulation | (μm) | (% nominal dose) |
| VII | 3.81 | 50.5 |
| VIII | 3.35 | 42.6 |

The weight loss up to 140° C. of Formulations VII and VIII were measured via TGA and were found to be 0.74% and 0.33%, respectively.

The Compound A content of Formulations VII and VIII were measured with HPLC-UV and are 101.2% and 99.5% of formula strength, respectively.

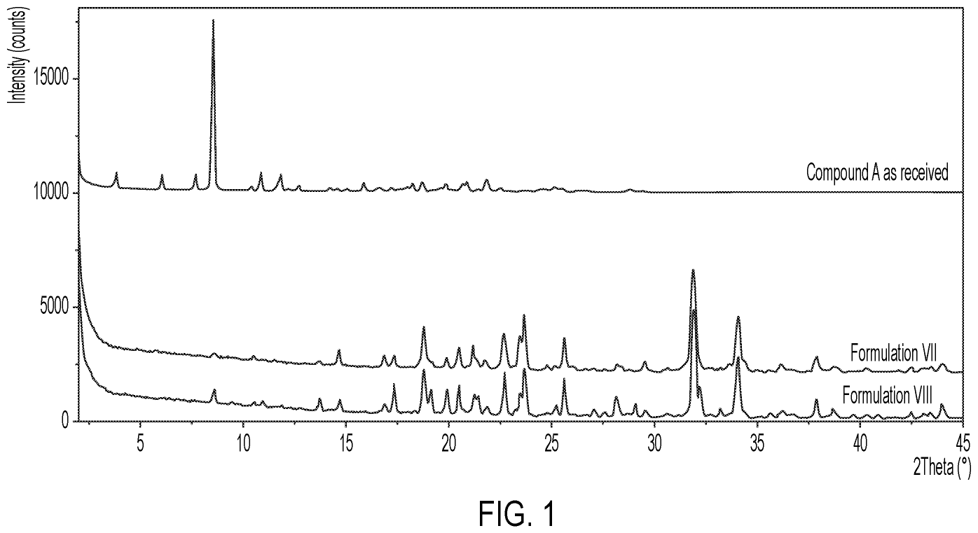

The crystallinity of Formulations VII and VIII were assessed via XRPD. Characteristic peaks from Compound A are observed in the diffraction pattern of both formulations, suggesting the milling or spray drying process does not affect the solid-state of Compound A. Additional peaks observed in the patterns correspond to the excipients in the formulations (FIG. 1).

### Example 3. Dry Powder Formulations of Nanocrystalline Compound a Containing Sodium Sulfate/Mannitol Manufactured at Increased Scale

### A. Powder Preparation.

The nanocrystalline Compound A was prepared by compounding 79.7 g of Compound A (Janssen, Lot A17KD2690) with 8.0033 g of polysorbate 80 (Sigma, Lot BCBX6159) in 197.0 g of water in an amber glass bottle. 5421 g of 200 μm YTZ milling media (Netzsch Zetabeads) was added to the chamber of the MiniCer stirred media mill (Netzsch GmbH, Selb Germany) and charged with an appropriate amount of water. The suspension was milled at 1000 RPM for 105 minutes before being collected. The final median particle size (Dv(50)) of the milled suspension was 134 nm.

Feedstock solutions were prepared and used to manufacture dry powders composed of nanocrystalline Compound A, polysorbate 80 and other additional excipients. Drug loads of 2 wt %, 5 wt % and 10 wt % Compound A, on a dry basis, were targeted. The feedstock solutions that were used to spray dry powders were made as follows. The required quantity of water was weighed into a suitably sized glass vessel, setting aside approximately 500 g of the required water for rinsing the suspension. The excipients were added to the water and the solution allowed to stir until visually clear. The Compound A-containing suspension was weighed in a beaker then added to the excipient solution, using the previously set-aside water to rinse the beaker. The feedstocks were stirred until visually homogenous, then spray-dried with continuous stirring throughout the run. Target feedstock masses were 6750 g, which supported manufacturing campaigns of 2 hours. Table 9 lists the components of the feedstocks used in preparation of the dry powders.

TABLE 9
Formulation IX-XI feedstock compositions

| | | | | Sodium | | |
|---|---|---|---|---|---|---|
| Formu- | Water | Compound | PS80 | sulfate | | Total |
| lation | (g) | A (g) | (g) | (g) | Mannitol | mass (g) |
| IX | 6547.5 | 3.9 | 0.4 | 99.0 | 99.0 | 6749.8 |
| X | 6547.6 | 9.8 | 1.0 | 95.8 | 95.8 | 6750.0 |
| XI | 6547.5 | 19.5 | 2.0 | 90.1 | 90.1 | 6749.2 |

PS80 = Polysorbate 80.

Dry powders of Formulations IX, X, and XI were manufactured from these feedstocks by spray drying on the Niro Mobile Minor Spray Dryer (GEA Process Engineering Inc., Columbia, MD) with cyclone powder collection. The system was run in open-loop (single pass) mode using nitrogen as the drying and atomization gas. Atomization of the liquid feed utilized a Niro nozzle 1.0 mm cap with a 2.5 mm separator. The aspirator of the system was adjusted to maintain the system pressure at −2.0″ water column.

The following spray drying conditions were followed to manufacture the dry powders. For Formulations IX, X, and XI, the liquid feedstock solids concentration was 30 g/kg, the process gas inlet temperature was 179° C. to 183° C., the process gas outlet temperature was 65° C., the drying gas flowrate was 80 kg/hr, the atomization gas flowrate was 365 g/min, the atomization gas backpressure at the atomizer inlet was 90 psig and the liquid feedstock flowrate was 50 g/min. The resulting dry powder formulations are reported in Table 10.

TABLE 10
Formulation IX-XI dry powder compositions, dry basis

| Formulation | Dry Powder Composition (w/w), dry basis |
|---|---|
| IX | 2.0% Compound A, 0.2% PS80, 48.9% sodium sulfate, |
| | 48.9% mannitol |
| X | 5.0% Compound A, 0.5% PS 80, 47.3% sodium sulfate, |
| | 47.3% mannitol |
| XI | 10.0% Compound A, 1.0% PS 80, 44.5% sodium sulfate, |
| | 44.5% mannitol |

### B. Powder Characterization.

The bulk particle size characteristics for the three formulations are found in Table 11. The span at 1 bar of 1.62, 1.56, and 1.50 for Formulations IX, X, and XI, respectively, indicates a relatively narrow size distribution. The 1 bar/4 bar dispersibility ratio of 0.99, 1.02 and 1.01 for Formulations IX, X, and XI respectively, indicate that they are relatively independent of dispersion energy, a desirable characteristic which allows similar particle dispersion across a range of dispersion energies.

TABLE 11
Formulation IX-XI bulk particle size

| | 0.5 bar | | 1 bar | | 4 bar | | |
|---|---|---|---|---|---|---|---|
| Formu- | Dv[50] | | Dv[50] | | Dv[50] | | 1 bar:4 bar |
| lation | (μm) | Span | (μm) | Span | (μm) | Span | Dv[50] ratio |
| IX | 1.89 | 1.57 | 1.78 | 1.62 | 1.80 | 1.61 | 0.99 |
| X | 2.01 | 1.51 | 1.87 | 1.56 | 1.83 | 1.56 | 1.02 |
| XI | 2.17 | 1.47 | 1.98 | 1.50 | 1.95 | 1.56 | 1.01 |

The aerodynamic particle size, fine particle fractions and fine particle doses measured and/or calculated with a Next Generation Impactor (NGI) are reported in Table 12. The fine particle dose for Formulations IX, X, and XI indicate a high percentage of the nominal dose which is filled into the capsule reaches the impactor stages (58.6%, 57.8% and 54.9%, respectively) and so would be predicted to be delivered to the lungs. The MMAD of Formulations IX, X, and XI were 3.00 μm, 3.12 μm and 3.02 μm, respectively, indicating deposition in the central and conducting airways.

TABLE 12
Formulation IX-XI aerodynamic particle size

| | MMAD | FPD < 5 μm |
|---|---|---|
| Formulation | (μm) | (% nominal dose) |
| IX | 3.00 | 58.6 |
| X | 3.12 | 57.8 |
| XI | 3.02 | 54.9 |

The weight loss up to 140° C. of Formulations IX, X, and XI were measured via TGA and were found to be 0.77%, 0.57% and 0.45%, respectively.

The Compound A content of Formulations IX, X, and XI were measured with HPLC-UV and are 93.9%, 94.5% and 93.9% of formula strength, respectively.

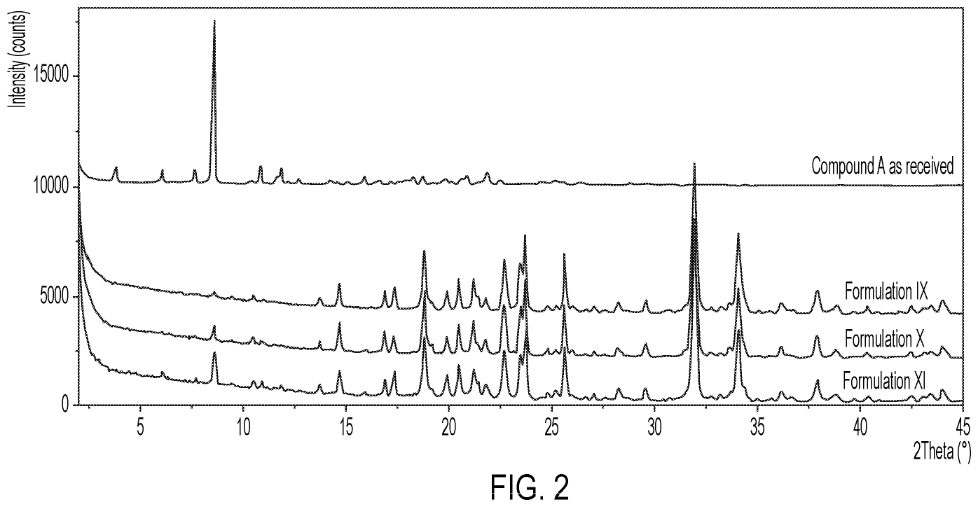

The crystallinity of Formulations IX, X, and XI were assessed via XRPD. Characteristic peaks from Compound A are observed in the diffraction pattern of all three formulations, suggesting the milling or spray drying process does not affect the solid-state of Compound A. Additional peaks observed in the patterns correspond to the excipients in the formulations (FIG. 2).

### Example 4. Dry Powder Formulation of Compound a for Use in Non-Clinical Toxicology Studies

### A. Powder Preparation.

The nanocrystalline Compound A used to support Formulation XII was prepared as follows. Two individual suspensions were formulated for milling. 25.00±0.10 g of Compound A (Janssen, Lot I15ED2302) was compounded with 2.50±0.01 g of polysorbate 80 (Sigma, Lot BCB59994) in 222.5 g of water and stirred until the Compound A was fully suspended. 502.5±3.0 g of 200 μm YTZ milling media (Tosoh Corp, Tokyo Japan) was added to the suspension. The suspensions were milled at 150 RPM for 28 hours before being collected. The two suspensions were pooled together after the target size was achieved. The final median particle size (Dv(50)) of the milled suspension was 129 nm.

A feedstock solution was prepared and used to manufacture a dry powder composed of nanocrystalline Compound A, polysorbate 80 and other additional excipients. Drug loading of 7.6 wt % Compound A, on a dry basis, was targeted. The feedstock solution that was used to spray dry the powder was made as follows. The required quantity of water was weighed into a suitably sized glass vessel, setting aside approximately 500 g of the required water for rinsing the suspension. The excipients were added to the water and the solution allowed to stir until visually clear. The Compound A-containing suspension was weighed in a beaker then added to the excipient solution, using the water set aside to rinse the beaker. The feedstock was stirred until visually homogenous then spray-dried with continuous stirring throughout the run. The mass of feedstock targeted was 10,000 g, which supported a manufacturing campaign of approximately 240 minutes. Table 13 lists the components of the feedstocks used in preparation of the dry powder.

TABLE 13
Formulation XII feedstock composition

| | | | | Sodium | | |
|---|---|---|---|---|---|---|
| | Water | Compound | Polysorbate | sulfate | Mannitol | Total |
| Formulation | (g) | A (g) | 80 (g) | (g) | (g) | mass (g) |
| XII | 9701 | 22.7 | 2.3 | 136.9 | 136.8 | 10,000 |

Formulation XII was manufactured from this feedstock by spray drying on the Niro Mobile Minor Spray Dryer (GEA Process Engineering Inc., Columbia, MD) with cyclone powder collection. The system was run in open-loop (single pass) mode using nitrogen as the drying and atomization gas. Atomization of the liquid feed utilized a Niro nozzle 1.0 mm cap with a 2.5 mm separator. The aspirator of the system was adjusted to maintain the system pressure at −2.0″ water column.

The following spray drying conditions were followed to manufacture the dry powder. The liquid feedstock solids concentration was 30 g/kg, the process gas inlet temperature was 170° C. to 178° C., the process gas outlet temperature was 70° C., the drying gas flowrate was 80 kg/hr, the atomization gas flowrate was 273 g/min, the atomization gas backpressure at the atomizer inlet was 34 to 35 psig and the liquid feedstock flowrate was 42 g/min. The resulting dry powder formulation is reported in Table 14.

TABLE 14
Formulation XII dry powder composition, dry basis

| Formulation | Dry Powder Composition (w/w), dry basis |
|---|---|
| XII | 7.6% Compound A, 0.8% polysorbate 80, 45.8% sodium |
| | sulfate, 45.8% mannitol |

### B. Powder Characterization.

The bulk particle size characteristics for Formulation XII is found in Table 15. The span at 1 bar of 1.55 indicates a relatively narrow size distribution. The 1 bar/4 bar dispersibility ratio of 1.03 indicates that the particle size distribution of the powder is relatively independent of dispersion energy, a desirable characteristic which allows similar particle dispersion across a range of dispersion energies.

TABLE 15
Formulation XII bulk particle size

| | 0.5 bar | | 1 bar | | 4 bar | | |
|---|---|---|---|---|---|---|---|
| Formu- | Dv[50] | | Dv[50] | | Dv[50] | | 1 bar:4 bar |
| lation | (μm) | Span | (μm) | Span | (μm) | Span | Dv[50] ratio |
| XII | 2.12 | 1.52 | 2.00 | 1.55 | 1.95 | 1.57 | 1.03 |

The aerodynamic particle size of Formulation XII was characterized for each dose group within each animal study (28-day rat and 28-day dog) via gravimetric cascade impaction (Marple 290 series, 296 configuration) at a flowrate of 3.0 L/min. The MMAD from the low, medium and high dose groups from Study 201-0127 (28-day rat study) were found to be 1.8 μm, 1.8 μm and 2.0 μm, respectively. The MMAD from the low, medium and high dose groups from Study 201-0128 (28-day dog study) were found to be 2.7 μm, 2.3 μm and 2.4 μm, respectively. The results indicate the powder is respirable and suitable for delivery to the lung.

The weight loss up to 140° C. of Formulation XII was measured via TGA and found to be 0.57%.

The Compound A content of Formulation XII was measured with HPLC-UV and found to be 95.4% of formula strength.

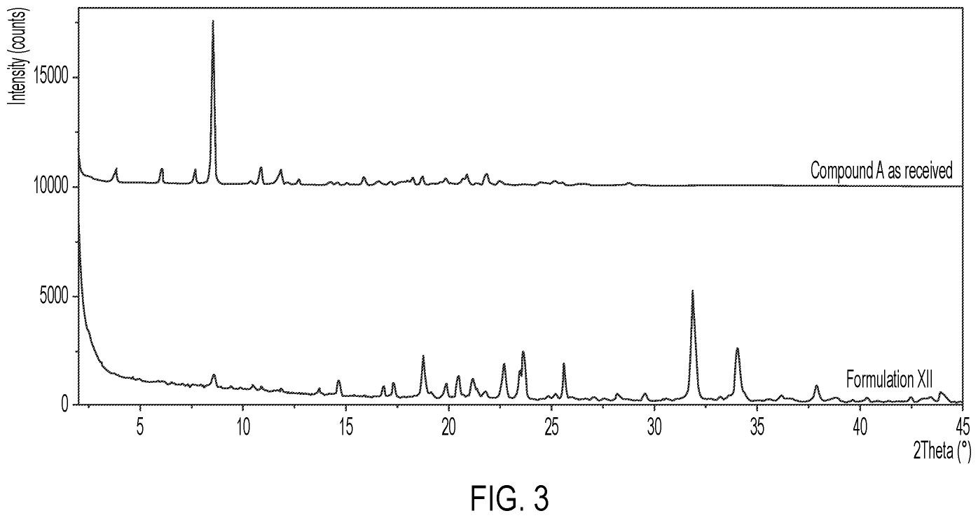

The crystallinity of Formulations XII was assessed via XRPD. Characteristic peaks from Compound A are observed in the diffraction pattern of the formulation, suggesting the milling or spray drying process does not affect the solid-state of Compound A. Additional peaks observed in the pattern correspond to the excipients in the formulation (FIG. 3).

### Example 5 (Reference Example). Lactose-Blend Formulations of Compound a for Use in Non-Clinical Toxicology Studies

### A. Powder Preparation.

An aliquot of pre-weighed lactose (Lactohale LH200) was added to the bowl of a Micro-Mixer. An amount of lactose that approximately equals the weight of magnesium stearate was added to the magnesium stearate container. This was stirred together manually using a stainless steel spoon for approximately one minute and added to the Micro-Mixer bowl. The magnesium stearate container was rinsed twice with a previously aliquoted portion of lactose, which was all added to the blend in the Micro-Mixer bowl. This blend (lactose containing 1% magnesium stearate) was mixed for approximately 5 minutes at 700 rpm. A quantity of this blend was added to the appropriate quantity of micronized Compound A (Janssen, Lot A12JD3143) to result in a 5% (w/w) Compound A concentration in the final blend. This was stirred together manually using a stainless steel spoon for approximately one minute and added to the Micro-Mixer bowl. The test material container was rinsed twice with a previously aliquoted portion of lactose, which is all added to the blend in the Micro-Mixer bowl. The lactose and Compound A were mixed twice for 5 minutes at 600 rpm. The blend was discharged into a labelled anti-static bag. The resulting dry powder formulation is reported in Table 16.

TABLE 16
Formulation XIII dry powder composition, dry basis

| Formulation | Dry Powder Composition (w/w), dry basis |
|---|---|
| XIII | 5% Compound A, 94% Lactohale LH200, 1% magnesium |
| | stearate |

### B. Powder Characterization.

The content uniformity of Formulation XIII was assessed by taking 10×100 mg samples from the top (x3), middle (x4) and bottom (x3) of the blend. The mean concentration of Compound A within the blend was found to be 49.5 mg/g with a coefficient of variation of 1.61%, representing a deviation from the mean of −1.1.

The aerodynamic particle size of Formulation XIII was characterized for each dose group within each animal study (28-day rat and 28-day dog) via cascade impaction (Marple 290 series, 296 configuration). The MMAD from the low, medium and high dose groups from Study AAO0100 (28-day rat study) were found to be 1.4 μm, 1.3 μm and 2.4 μm, respectively. The MMAD from the low, medium and high dose groups from Study AAO0097 (28-day dog study) were found to be 1.0 μm, 1.3 μm and 1.4 μm, respectively. The results indicate the powder is respirable and suitable for delivery to the lung.

### Example 6: 28-Day PK Studies of Varying Compound a Formulations

Plasma and lung tissue homogenate concentrations of Compound A were measured using appropriately qualified or validated liquid chromatography/tandem mass spectrometry (LC-MS/MS) methods.

The kinetics of Compound A as a lactose blend (Formulation XIII) and as Formulation XII) were assessed as part of 28-Day inhalation toxicology studies. The specific studies and achieved doses are detailed in Table 17.

TABLE 17
Compound A Pharmacokinetic Studies and Key Findings

| Report | | | | |
|---|---|---|---|---|
| Number | Study Title | Species | Achieved Doses | Key Findings |
| AAO0100 | Formulation XIII: Toxicity | Rat | 9.16, 30.3, 99.9 and | Less than dose proportional |
| | Study by Inhalation (Dry | | 2930 μg/kg/day | plasma exposure |
| | Powder) Administration to | | | 2.6- to 5-fold plasma |
| | Wistar Han Rats for 4 Weeks | | | accumulation |
| | Followed by a 4 Week | | | Steady state plasma by Day |
| | Recovery Period | | | 14 |
| | | | | Greater than dose |
| | | | | proportional lung exposure |
| | | | | High lung exposure and |
| | | | | lung retention through 28 |
| | | | | day recovery |
| AAO0097 | Formulation XIII: Toxicity | Dog | Inhalation, 10.9, 30.5 | Broadly dose proportional |
| | Study by Inhalation (Dry | | 108 and 315 μg/kg/day | plasma exposure |
| | Powder) Administration to | | for 28 days | 2.0- to 4.2-fold plasma |
| | Beagle Dogs for 4 Weeks | | | accumulation |
| | Followed by a 4 Week | | | Steady state plasma by Day |
| | Recovery Period | | | 14 |
| | | | | Broadly dose proportional |
| | | | | lung exposure |
| | | | | High lung exposure and |
| | | | | lung retention through 28 |
| | | | | day recovery |
| 201-0127 | Formulation XII: Toxicity | Rat | Inhalation, 53.9, 122 | Broadly dose proportional |
| | Study by Inhalation | | and 259 μg/kg/day for | plasma exposure |
| | Administration to Sprague- | | 28 days | 1.0- to 1.6-fold plasma |
| | Dawley Rats for 4 Weeks | | | accumulation |
| | Followed by a 4 Week | | | Steady state plasma by Day |
| | Recovery Period | | | 14 |
| | | | | Broadly dose proportional |
| | | | | lung exposure |
| | | | | 1.2- to 2.7-fold lung |
| | | | | accumulation |
| | | | | Steady state lung by Day 14 |
| | | | | High lung exposure but |
| | | | | BLOQ by recovery day 7 |
| 201-0128 | Formulation XII: Toxicity | Dog | Inhalation, 37.4, 77.1 | Broadly dose proportional |
| | Study by Inhalation | | and 128 μg/kg/day for | plasma exposure |
| | Administration to Beagle | | 28 days | 1.5- to 2.0-fold plasma |
| | Dogs for 4 Weeks Followed | | | accumulation |
| | by a 4 Week Recovery | | | Steady state plasma by Day |
| | Period | | | 14 |
| | | | | Broadly dose proportional |
| | | | | lung exposure |
| | | | | 1.2- to 2.7-fold lung |
| | | | | accumulation |
| | | | | Steady state lung by Day 14 |
| | | | | High lung exposure but |
| | | | | BLOQ by recovery day 28 |

### Formulation XIII (Lactose Blend) Versus Formulation XII Toxicokinetic Comparison

The exposure kinetics of Formulation XIII in the rat 28 day study indicated that steady state for Compound A was likely achieved by Day 14. Significant systemic accumulation of Compound A occurred through the first 14 days, with 2.6 to 5.0-fold accumulation across the doses used by that point but little or no additional accumulation thereafter. Systemic exposure to Compound A in rats with Formulation XII was generally lower than that after Formulation XIII on Day 1 at approximately equivalent doses for reasons that are unclear. In addition, systemic accumulation of Compound A after Formulation XII dosing was substantially lower relative to Formulation XIII (lactose blend) with accumulation of 1.0 to 1.6-fold across the doses used over the course of 28 days of dosing.

Lung exposure in rats across both formulations was quite different. Trough levels (24 h post dose) for Formulation XIII (lactose blend) were not determined in the 28 day study but were assessed on Day 1, 28 and 182 for a 6-month study, as well as 24 h after the Day 28 dose of the 28-day study. Given that the Day 28 values were similar across both the 28 day and 6 month Formulation XIII (lactose blend) studies, it can be surmised that the accumulation seen over 28 days in the 6 month study is the same as that in the 28-day study and demonstrated substantial accumulation relative to Day 1 of 4.9 to 11.4-fold across the doses used, suggesting retention of a substantial amount of the Compound A dose in the lung. For Formula XII, samples were collected at trough on Days 1, 14 and 28 and showed much lower lung levels of Compound A relative to Formulation XIII (lactose blend) at trough on Days 1 and 28. In contrast to Formulation XIII (lactose blend), there was less accumulation over the course of the 28 days of dosing with Formulation XII, with only 1.2 to 2.7-fold accumulation across the doses used.

In dogs dosed with Formulation XIII (lactose blend) for 28 days, the systemic accumulation of Compound A through 14 and 28 days was 2.1 to 4.2-fold at 14 days across the doses used and 2.0 to 4.3-fold on Day 28 across the doses used, indicating that steady state was likely achieved by Day 14. With Formulation XII dosing, the systemic exposure to Compound A was slightly higher than that with the lactose blend (Formulation XIII) at approximately equivalent doses, but accumulation of Compound A over 28 days was less than that seen for the lactose blend (Formulation XIII), ranging from 1.4 to 2.1-fold across the doses used over 28 days of dosing.

When comparing lung exposure in the dog, only terminal trough samples could be collected 24 h after the last dose. Formulation XII dosing resulted in Day 28 trough levels substantially lower than those seen with the lactose blend (Formulation XIII) suggesting that, at approximately equivalent doses, the level of lung accumulation with Formulation XII was likely to have been substantially lower than that with Formulation XIII (lactose blend), as was seen in the rat.

From all the pharmacokinetic data it is clear that, for approximately equivalent delivered doses of Compound A, administration of Formulation XII results in lower systemic and lung accumulation and more rapid clearance of Compound A after the end of dosing in comparison to administration of the lactose blend (Formulation XIII).

### Example 7: Toxicity Studies of Different Formulations of Compound A

Individual achieved Compound A doses and for the 28-day studies with Formulation XIII (lactose blend) and Formulation XII are summarized in Table 18.

TABLE 18
Achieved Doses in the 28-Day GLP Repeat Dose Rat Studies

| | | Target | Achieved |
|---|---|---|---|
| | | Delivered Dose | Delivered Dose |
| Study | Group | (μg/kg/day) | (μg/kg/day) |
| Formulation XIII: Toxicity Study by | 1 | 0 (placebo) | 0 |
| Inhalation (Dry Powder) Administration to | 2 | 10 | 9.16 |
| Wistar Han Rats for 4 Weeks Followed by | 3 | 30 | 30.3 |
| a 4 Week Recovery Period | 4 | 100 | 99.9 |
| (AAO0100) | 5 | 3000 | 2930 |
| Formulation XII: Toxicity Study by | 1 | 0 (air control) | 0 |
| Inhalation Administration to Sprague- | 2 | 0 (placebo) | 0 |
| Dawley Rats for 4 Weeks Followed by a 4 | 3 | 60 | 53.9 |
| Week Recovery Period | 4 | 120 | 122 |
| (201-0127) | 5 | 240 | 259 |
| Formulation XIII: Toxicity Study by | 1 | 0 (placebo) | |
| Inhalation (Dry Powder) Administration to | 2 | 10 | 10.9 |
| Beagle Dogs for 4 Weeks Followed by a 4 | 3 | 30 | 30.5 |
| Week Recovery Period | 4 | 100 | 108 |
| (AAO0097) | 5 | 300 | 315 |
| Formulation XII: Toxicity Study by | 1 | 0 (air control) | 0 |
| Inhalation Administration to Beagle Dogs | 2 | 0 (placebo) | 0 |
| for 4 Weeks Followed by a 4 Week | 3 | 40 | 37.4 |
| Recovery Period | 4 | 80 | 77.1 |
| (201-0128) | 5 | 120 | 128 |

All of the nonclinical toxicology studies were performed by the same nonclinical CRO, Envigo (Huntingdon Life Sciences), Huntingdon, Cambridgeshire, UK. In addition, the same consultant pathologist performed the peer review of all studies. The target delivered doses for the Formulation XIII (lactose blend) and Formulation XII studies were chosen based on the required clinical safety margins and so differ across both studies, however, there is sufficient overlap in achieved delivered doses to make a reasonable comparison of the microscopic pathology.

In the rat, aggregates of mixed inflammatory cells (primarily macrophages, lymphocytes and granulocytes) adjacent to the terminal bronchioles and alveolar ducts were observed in the majority of rats receiving 99.9 μg/kg/day Compound A as Formulation XIII (lactose blend). These aggregates were all graded minimal or slight in terms of severity and appeared to reside in the interstitial compartment of the lung rather than the airways.

Aggregates of mixed inflammatory cells were also observed in a minority of rats receiving 259 μg/kg/day Compound A as Formulation XII for 28 days but not at 122 μg/kg/day. The cellular composition and location of the aggregates was similar to those present in rats receiving Formulation XIII (lactose blend). However, these aggregates were all of minimal severity and distributed sparsely with only one lung lobe affected in some individuals. No other features were observed in the lungs of rats given Formulation XII.

In the dog, aggregates of mixed inflammatory cells (primarily macrophages, lymphocytes and granulocytes) adjacent to the terminal bronchioles and alveolar ducts were observed in dogs at ≥30.5 μg/kg/day Compound A as Formulation XIII (lactose blend) for 28 days. The incidence and severity of this finding were dose related. At 315 μg/kg/day Compound A as Formulation XIII (lactose blend), the aggregates were graded as moderate in the majority of dogs and were relatively large in size and diffusely distributed across all lung lobes but minimal to slight severity at 108 μg/kg/day.

Aggregates of mixed inflammatory cells were also seen in the majority of dogs given 124 μg/kg/day Compound A as Formulation XII for 28 days. The cellular composition and location of the aggregates was similar to those present in rats receiving Compound A as Formulation XIII (lactose blend). However, these aggregates were mostly of minimal severity and distributed sparsely within the lung.

Overall, the inhalation administration of Formulation XIII (lactose blend) or Formulation XII to rats and dogs for 4 weeks was associated with the development of mixed inflammatory cell aggregates in the lungs at high dose with both formulations. The characteristic change with both formulations was the presence of inflammatory cell aggregates in close proximity to terminal bronchioles and alveolar ducts. No evidence of degenerative or necrotic changes in the lung was noted with either formulation, and, although the type of pathology was similar between the two formulations, over the range of doses examined, both the incidence and severity of these findings was lower with Formulation XII at approximately equivalent doses. The mixed inflammatory cell aggregates occurred in 13/20 rats at 99.9 μg/kg/day with the lactose blend (Formulation XIII), reaching a severity grading of slight in one animal, whereas only 6/20 rats showed a similar response with Formulation XII at 259 μg/kg/day, with all at minimal severity, and no mixed inflammatory cell aggregates were observed at 122 μg/kg/day with Formulation XII. Similarly, mixed inflammatory cell aggregates occurred in 5/6 dogs at 108 μg/kg/day with the Formulation XIII (lactose blend), reaching a severity grading of slight in 3 animals and the same finding was observed in all 6 dogs at 315 μg/kg/day with Formulation XIII (lactose blend), reaching a severity grading of moderate in 4 animals. This contrasts to the findings in dogs dosed with Formulation XII, where mixed inflammatory cell aggregates occurred in 5/6 animals at 124 μg/kg/day but only reached a severity grading of slight in one animal. In addition, in both species, the findings were only partially reversed after the recovery period in animals dosed with the Formulation XIII lactose blend, whereas full recovery was seen in all dose groups in animals dosed with Formulation XII.

Although the type of lung pathology was similar between the two formulations, over the range of doses used, the incidence and severity of these findings was generally lower with Formulation XII.

### Example 8: Dry Powder Formulation of Compound a (Formulation XI) for Use in Chronic Non-Clinical Studies

### A. Powder Preparation.

The nanocrystalline Compound A used to support Formulation XI for chronic non-clinical studies was prepared as follows. Two individual suspensions were formulated for milling. 200.±1.00 g of Compound A (Janssen, Lot A17KD2690) was compounded with 20.0±0.10 g of polysorbate 80 (Sigma, Lot BCCB4769) in 780.0 g of water and stirred until the Compound A was fully suspended. The suspensions were milled using a Netzsch MiniCer (NETZSCH GmbH, Selb Germany) at a tip speed of 8 m/s (2389 RPM) with 1160±5.8 g of 500 μm YTZ milling media (Glen Mills, Clifton, NJ) for 90 minutes before being collected. The two suspensions were pooled together after the target size was achieved. The final median particle size (Dv(50)) of the milled suspension was 260 nm. The crystallinity of the nanocrystalline suspension was assessed via XRPD. Characteristic peaks from Compound A are observed in the diffraction pattern of the suspension, suggesting the milling process does not affect the solid-state of Compound A.

Feedstock suspensions were prepared and used to manufacture a dry powder comprising nanocrystalline Compound A, polysorbate 80 and other additional excipients. Drug loading of 10.0 wt % Compound A, on a dry basis, was targeted. The feedstock suspension that was used to spray dry the powder was made as follows. The desired quantity of water was weighed into a suitably sized stainless-steel tank, setting aside approximately 500 g of the water for rinsing the suspension. The excipients were added to the water and the solution allowed to stir until visually clear. The Compound A-containing suspension was weighed in a beaker then added to the excipient solution, using the water set aside to rinse the beaker. The feedstock was stirred until visually homogenous then spray-dried with continuous stirring throughout the run. The mass of feedstock targeted was 19,200 g, which supported a daily manufacturing time of approximately 480 minutes. Four days of spray drying were completed with a feedstock being prepared for each day. Table 19 lists the components of the feedstocks used in preparation of the dry powder Formulation XI for use in chronic non-clinical studies.

TABLE 19
Formulation XI feedstock compositions

| | | | | Sodium | | |
|---|---|---|---|---|---|---|
| | Water | Compound | Polysorbate | sulfate | Mannitol | Total mass |
| Formulation | (g) | A (g) | 80 (g) | (g) | (g) | (g) |
| XI (feedstock a) | 18625 | 57.624 | 5.7624 | 256.61 | 256.43 | 19201 |
| XI (feedstock b) | 18615 | 57.664 | 5.7664 | 256.74 | 256.35 | 19192 |
| XI (feedstock c) | 18695 | 57.756 | 5.7756 | 256.57 | 256.40 | 19272 |
| XI (feedstock d) | 18655 | 57.724 | 5.7724 | 256.44 | 256.59 | 19232 |

Formulation XI was manufactured from these feedstocks by spray drying on the Niro Mobile Minor Spray Dryer (GEA Process Engineering Inc., Columbia, MD) with dual-cyclone powder collection. The system was run in open-loop (single pass) mode using nitrogen as the drying and atomization gas. Atomization of the liquid feed utilized a Spraying Systems Two-Fluid Nozzle 2050/120.

The following spray drying conditions were followed to manufacture the dry powder. The liquid feedstock solids concentration was 30 g/kg, the process gas inlet temperature was 114° C. to 123° C., the process gas outlet temperature was 65° C., the drying gas flowrate was 180 kg/hr, the atomization gas flowrate was 290 g/min, the atomization gas backpressure at the atomizer inlet was 70 psig and the liquid feedstock flowrate was 40 g/min. The resulting dry powder formulation XI is reported in Table 20.

TABLE 20
Formulation XI dry powder composition, dry basis

| Formulation | Dry Powder Composition (w/w), dry basis |
|---|---|
| XI | 10.00% Compound A, 1.00% polysorbate 80, 44.50% |
| | sodium sulfate, 44.50% mannitol |

### B. Powder Characterization

The bulk particle size characteristics for Formulation XI were tested on a Malvern 3000 with Aero S dry powder feeder, measured at 4 bar. The bulk particle size characteristics for Formulation XI are found in Table 21. The span at 4 bar of 2.09 indicates a relatively narrow size distribution.

TABLE 21
Formulation XI bulk particle size

4 bar

| | Dv[50] | |
|---|---|---|
| Formulation | (μm) | Span |
| XI | 1.71 | 2.09 |

The aerodynamic particle size and fine particle doses measured and/or calculated with a Next Generation Impactor (NGI) are reported in Table 22. The fine particle dose for Formulation XI indicates a high percentage of the nominal dose which is filled into the capsule reaches the impactor stages (57%) and so would be predicted to be delivered to the lungs. The MMAD of Formulation XI was 2.30 μm, indicating deposition in the central and conducting airways.

TABLE 21
Formulation XI aerodynamic particle size

| | MMAD | FPD < 5 μm |
|---|---|---|
| Formulation | (μm) | (% nominal dose) |
| XI | 2.30 | 57 |

The water content of Formulation XI (for chronic non-clinical studies) was measured via Karl Fischer and found to be 0.65%.

The Compound A content of Formulation XI (for chronic non-clinical studies) was measured with HPLC-UV and found to be 101.3% of formula strength.

### Example 9: Non-Clinical Plasma Accumulation Pharmacokinetic Studies and Toxicology Studies with Exemplary Non-Lactose Dry Powder Formulation

Non-clinical plasma accumulation studies were carried out using non-lactose dry powder formulation XI (i.e., Formulation XI for chronic non-clinical studies prepared in Example 8), containing Compound A, in rats and dogs. Data from this study was contrasted with data obtained in analogous studies using a lactose-blend formulation (Formulation XIII). Clinical studies (i.e., in humans) were also carried out and described in more detail in Example 11 below.

Results from plasma accumulation studies in rats exposed to a lactose-blend formulation (Formulation XIII) or a non-lactose dry powder formulation (Formulation XI) of Compound A over a period of six months is provided in Table 22 below.

TABLE 22
6-month accumulation study in rats with lactose-
blend and non-lactose dry powder formulation.

| | | | | | Non-Lactose Dry Powder | | |
|---|---|---|---|---|---|---|---|
| | Lactose Blend (Formulation XIII) | | | | (Formulation XI) | | |
| | Delivered dose (μg/kg/day) | | | | Delivered dose (μg/kg/day) | | |
| | 10.5* | 32.4* | 104* | 1120* | 58.7 | 124 | 256 |
| Day 1 | | | | | | | |
| AUC_{0-24 h} | 5.7 | 11.4 | 23.75 | 149 | 8.6 | 17.2 | 34.0 |
| (h*ng/mL) | | | | | | | |
| Cₘₐₓ (ng/mL) | 0.744 | 1.42 | 3.19 | 21.1 | 3.07 | 6.28 | 11.1 |
| Lung Trough | 320 | 1,475 | 4,332 | 33,034 | 72.0 | 159 | 336 |
| (ng/g) | | | | | | | |
| Day 91 | | | | | | | |
| AUC_{0-24 h} | 7.96 | 19.9 | 67.6 | 618.5 | 10.1 | 28.1 | 64.3 |
| (h*ng/mL) | | | | | | | |
| Cₘₐₓ (ng/mL) | 0.474 | 1.2 | 5.16 | 37.25 | 1.42 | 2.91 | 6.96 |
| DN AUC | 3.8 | 1.9 | 2.6 | 3.5 | 1.6 | 1.0 | 1.37 |
| Accumulation | | | | | | | |
| Day 182 | | | | | | | |
| AUC_{0-24 h} | 7.6 | 18.8 | 69.8 | 641 | 23.6 | 33.4 | 113 |
| (h*ng/mL) | | | | | | | |
| Cₘₐₓ (ng/mL) | 0.441 | 1.13 | 4.31 | 37.7 | 2.03 | 2.76 | 8.22 |
| Lung Trough | 2,197 | 5,757 | 26,850 | 598,334 | 1170 | 1550 | 7540 |
| (ng/g) | | | | | | | |
| DN AUC | 3.6 | 2.1 | 2.6 | 3.9 | 2.02 | 1.33 | 1.89 |
| Accumulation | | | | | | | |

*Simple average of data for each sex; DN = dose normalized.

Results from plasma accumulation studies in dogs exposed to a lactose-blend formulation (Formulation XIII) over a period of 13 weeks, or a non-lactose dry powder formulation (Formulation XI) of Compound A over a period of nine months, is provided in Table 23 below.

TABLE 23
Accumulation study in dogs with lactose-blend and non-lactose formulation.

| | | | | | Non-Lactose Dry Powder | | |
|---|---|---|---|---|---|---|---|
| | Lactose Blend (Formulation XIII) | | | | (Formulation XI) | | |
| | Delivered dose (μg/kg/day) | | | | Delivered dose (μg/kg/day) | | |
| | 10.2 | 31.6 | 105 | 311 | 37.4 | 73.1 | 109 |
| Day 1 | | | | | | | |
| AUC_{0-24 h} | NC | NC | 4.9 | 17.2 | 5.71 | 9.64 | 18.3 |
| (h*ng/mL) | | | | | | | |
| Cₘₐₓ (ng/mL) | NC | 0.265 | 0.601 | 2.72 | 2.76 | 2.28 | 6.4 |
| Day 91 | | | | | | | |
| AUC_{0-24 h} | 3.7 | 9.4 | 35.8 | 78.4 | 12.2 | 19.5 | 31.4 |
| (h*ng/mL) | | | | | | | |
| Cₘₐₓ (ng/mL) | 0.238 | 0.74 | 2.9 | 7.04 | 1.38 | 2.09 | 3.81 |
| DN AUC | NC | NC | 6.8 | 4.1 | 2.2 | 2.08 | 1.87 |
| Accumulation | | | | | | | |
| Lung Trough | 1,773 | 7,383 | 26,263 | 77,250 | | | |
| (ng/g) | | | | | | | |
| Day 273 | | | | | | | |
| AUC_{0-24 h} | | | | | 14.7 | 24.6 | 38.9 |
| (h*ng/mL) | | | | | | | |
| Cₘₐₓ (ng/mL) | | | | | 1.58 | 2.73 | 4.48 |
| Lung Trough | | | | | 1,210 | 2,400 | 4,400 |
| (ng/g) | | | | | | | |
| DN AUC | | | | | 2.02 | 2.30 | 2.03 |
| Accumulation | | | | | | | |

DN = dose normalized

Broadly dose-proportional exposure was observed for both formulations, in both rats and dogs. Substantially higher lung levels and lung:plasma ratio was observed in the lactose blend relative to the non-lactose dry powder after chronic dosing. In rats that received all doses of Formulation XI, lung exposure to Compound A was consistently absent at four weeks of recovery, but was consistently measurable at 4 weeks of recovery in rats that received the lactose blend, and up to 8 weeks of recovery in rats that received high doses of the lactose blend. Similarly, lung exposure to Compound A was consistently absent at 6 weeks of recovery in dogs that received Formulation XI at all doses, but was consistently measurable at 7 weeks of recovery in dogs that received high doses of the lactose blend.

The results demonstrate that plasma accumulation was about 3-fold higher in the lactose blend formulation compared to the non-lactose dry powder formulation, at approximately equal nominal doses, or in other words, repeated administration of a non-lactose dry powder formulation of the present disclosure can lead to three times less accumulation than a lactose-blend at similar doses.

Plasma accumulation appeared to increase with increasing dose duration in the animal studies using the lactose-blend formulation, but not when using the non-lactose dry powder formulation which had relatively consistent accumulation even with up to 6 and 9 month-long periods of dosing. Additionally, clearance from the plasma after the end of dosing was generally faster with non-lactose dry powder formulation dosing, which was consistent with the lower accumulation observed.

In the clinical studies, discussed in more detail in Example 11, steady state was not reached by 14 days with the lactose blend formulation, and steady state was projected not to occur until at least 28 days. On the other hand, clinical data with the non-lactose dry powder formulation indicates that steady state was reached between 7 to 14 days.

Peak plasma concentration (Cₘₐₓ) at Day 1 in the rats that received a nominal dose of 124 μg/kg of Formulation XI (non-lactose dry powder) was 6.28 ng/mL, which is almost double the Cₘₐₓ measured at Day 1 in rats that received a similar nominal dose (104 μg/kg) of the lactose blend formulation (Cₘₐₓ=3.19 ng/mL). Also, Cₘₐₓ at Day 1 in the dogs that received a nominal dose of 109 μg/kg of Formulation XI (non-lactose dry powder) was 6.4 ng/mL, which was over ten times higher than the Cₘₐₓ measured in dogs at Day 1 that received a similar nominal dose (105 μg/kg) of the lactose blend, which was only 0.601 ng/mL. Additionally, at time points after Day 91 in these studies the Cₘₐₓ recorded in animals that received the lactose blend were significantly larger than in the animals receiving Formulation XI. Without wishing to be bound by theory, it is believed that initial doses of the lactose blend accumulate in the lungs and become tissue-bound, which not only blunts the Cₘₐₓ initially but later contributes to delayed and long-lasting accumulation-driven exposure, which is hard to control. These problems are not observed when the non-lactose formulation of the present disclosure is administered, as it is rapidly dissolved and does not substantially accumulate in lung tissue.

In summary, these data indicate that lactose blend formulations of Compound A have poorly controlled exposure kinetics, resulting in initially low systemic exposure, with accumulation that increases with increasing duration of dosing, and exhibits slow clearance. Non-lactose dry powder formulations of the present disclosure (e.g., Formulation XI), on the other hand, have less overall accumulation and reach steady state and clear more quickly, allowing for more controlled and predictable exposure regardless of dose duration. Further, to achieve a therapeutic level of Compound A in systemic circulation, it is necessary to administer much larger amounts of the lactose blend to the lung, relative to the non-lactose dry powders of the present disclosure, which results in a significantly higher lung exposure and also exacerbates risks associated with accumulation that occurs in the lactose blend.

### Example 10. Exemplary Dry Powder Formulations of Nanocrystalline Compound a Manufactured for Use in Clinical Studies

### A. Powder Preparation.

The nanocrystalline Compound A used to support Formulations X and XI for clinical studies was prepared as follows. One suspension was formulated for milling. 200.±1.00 g of Compound A (Janssen, Lot A17KD2690) was compounded with 20.0±0.10 g of polysorbate 80 in 780.0 g of water and stirred until the Compound A was fully suspended. The suspensions were milled using a DynoMill (Willy A. Bachofen, Muttenz, Switzerland) at a tip speed of 8 m/s (2389 RPM) with 1160±5.8 g of 500 μm YTZ milling media (Glen Mills, Clifton, NJ) for 90 minutes before being collected. The final median particle size (Dv(50)) of the milled suspension was 190 nm.

Feedstock suspensions were prepared and used to manufacture dry powders composed of nanocrystalline Compound A, polysorbate 80 and other additional excipients. Drug loads of 5 wt % and 10 wt % Compound A, on a dry basis, were targeted. The feedstock solutions that were used to spray dry powders were made as follows. The desired quantity of water was weighed into a suitably sized stainless-steel tank, setting aside approximately 750 g of the water for rinsing the suspension. The excipients were added to the water and the solution allowed to stir until visually clear. The Compound A-containing suspension was weighed in a beaker then added to the excipient solution, using the previously set-aside water to rinse the beaker. The feedstocks were stirred until visually homogenous, then spray-dried with continuous stirring throughout the run. Target feedstock masses were 25000 g, which supported manufacturing campaigns of approximately 10 hours. Table 24 lists the components of the feedstocks used in preparation of the dry powders.

TABLE 24
Formulation X and XI feedstock compositions

| | | | | Sodium | | |
|---|---|---|---|---|---|---|
| | Water | Compound | Polysorbate | sulfate | Mannitol | Total mass |
| Formulation | (g) | A (g) | 80 (g) | (g) | (g) | (g) |
| X | 24246.6 | 37.5 | 3.75 | 354.4 | 354.4 | 24996.7 |
| XI | 24252.5 | 75.0 | 7.50 | 333.8 | 333.8 | 25002.3 |

Dry powders of Formulations X and XI for clinical studies were manufactured from these feedstocks by spray drying on a custom-built GMP spray dryer (Lonza AG, Bend Oregon) with dual-cyclone powder collection. The system was run in open-loop (single pass) mode using nitrogen as the drying and atomization gas. Atomization of the liquid feed utilized a Spraying Systems Two-Fluid Nozzle 2050/120.

The following spray drying conditions were followed to manufacture the dry powders. For Formulations X and XI, the liquid feedstock solids concentration was 30 g/kg, the process gas inlet temperature was 107° C. to 116° C., the process gas outlet temperature was 65° C., the drying gas flowrate was 180 kg/hr, the atomization gas flowrate was 290 g/min, the atomization gas backpressure at the atomizer inlet was 70 psig and the liquid feedstock flowrate was 40 g/min. The resulting dry powder formulations are reported in Table 25.

TABLE 25
Formulation X and XI (for clinical studies)
dry powder compositions, dry basis

| Formulation | Dry Powder Composition (w/w), dry basis |
|---|---|
| X | 5.0% Compound A, 0.5% PS 80, 47.3% sodium sulfate, |
| | 47.3% mannitol |
| XI | 10.0% Compound A, 1.0% PS 80, 44.5% sodium sulfate, |
| | 44.5% mannitol |

### B. Powder Characterization.

The bulk particle size characteristics for Formulations X and XI for clinical studies are found in Table 26. The span at 4 bar of 2.07, and 2.13 for Formulations X, and XI, respectively, indicates a relatively narrow size distribution.

TABLE 26
Formulation X and XI for clinical studies bulk particle size

4 bar

| | Dv[50] | |
|---|---|---|
| Formulation | (μm) | Span |
| X | 1.58 | 2.07 |
| XI | 1.70 | 2.13 |

The aerodynamic particle size and fine particle doses measured and/or calculated with a Next Generation Impactor (NGI) are reported in Table 27. The fine particle dose for Formulations X and XI for clinical studies indicate a high percentage of the nominal dose which is filled into the capsule reaches the impactor stages (67% and 58%, respectively) and so would be predicted to be delivered to the lungs. The MMAD of Formulations X and XI for clinical studies were 2.38 μm and 2.46 μm, respectively, indicating deposition in the central and conducting airways.

TABLE 27
Formulation X-XI for clinical studies aerodynamic particle size

| | MMAD | FPD < 5 μm |
|---|---|---|
| Formulation | (μm) | (% nominal dose) |
| X | 2.38 | 67 |
| XI | 2.46 | 58 |

The water content of Formulations X and XI for clinical studies were measured via Karl Fisher and were found to be 0.54% and 0.51%, respectively.

The Compound A content of Formulations X and XI for clinical studies were measured with HPLC-UV and are 98.2% and 98.3% of formula strength, respectively.

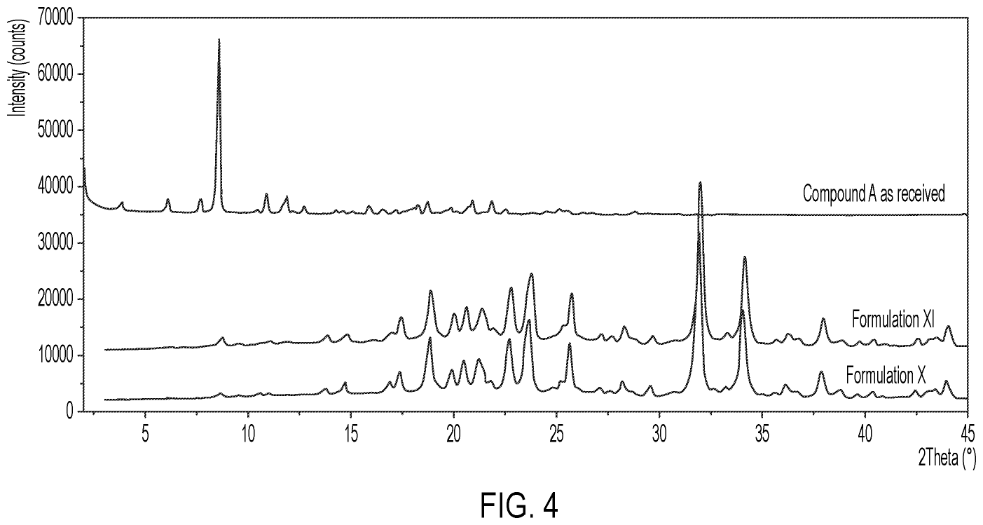

The crystallinity of Formulations X and XI for clinical studies were assessed via XRPD, and relevant XRPD patterns are provided in FIG. 4. Characteristic peaks from Compound A are observed in the diffraction pattern of each formulation, suggesting the milling or spray drying process does not affect the solid-state of Compound A. Additional peaks observed in the patterns correspond to the excipients in the formulations.

### Example 11: Clinical Studies with Non-Lactose Dry Powder Formulation and Modelled Clinical Data

### Clinical Trial

A Phase 1b clinical study of non-lactose formulations of the present disclosure, administered as dry powders for inhalation, was carried out in subjects with stable Chronic Obstructive Pulmonary Disease (COPD).

The phase 1b trial was designed as a randomized, double-blind, placebo-controlled, 3-way crossover study to assess the safety, tolerability, and pharmacokinetics of repeated once-daily doses of a non-lactose formulation or placebo for 14 days, in adult subjects with stable COPD. A total of 18 subjects were enrolled. Safety and tolerability as well as systemic pharmacokinetics were evaluated. The non-lactose dry powder formulation was administered at nominal doses of 250 μg or 500 μg, using Formulations X or XI, respectively, which were prepared according to Example 10.

Both doses of the non-lactose blend dry powder were safe and well-tolerated, and there were no observed safety signals or treatment emergent adverse findings related to the dry powders.

The pharmacokinetic parameters obtained during this study (at 500 μg nominal dose) are summarized below in Table 28, in addition to comparative data obtained using a lactose blend formulation.

TABLE 28
Pharmacokinetics from clinical studies with non-lactose and lactose blend formulations.

| | Non-Lactose Dry Powder | | | | Lactose Blend | | | | |
|---|---|---|---|---|---|---|---|---|---|
| | (Formulation XI) | | | | (Formulation XIII) | | | | |
| | Dose | Cₘₐₓ | AUC_{(0-24 h)} | AUC | | Dose | Cₘₐₓ | AUC_{(0-24 h)} | AUC |
| Day | (μg) | (pg/mL) | (pg*h/mL) | accum | Day | (μg) | (pg/mL) | (pg*h/mL) | accum |
| 1 | 500 | 170 | 1960 | — | 1 | 500 | 66.8 | 835 | — |
| 7 | 500 | 328 | 3640 | <2.1 | 7 | 500 | 256 | 4120 | 4.9 |
| 14 | 500 | 347 | 3960 | <2.1 | 14 | 500 | 379 | 5490 | 6.6 |

This data indicates that the non-lactose dry powder formulations resulted in low and consistent systemic exposure when administered via oral inhalation, which was consistent with findings made in preclinical studies discussed in Example 9. Notably, systemic absorption was rapid, with a median Tₘₐₓ less than or equal to 0.75 hours post dose, on all days and at all doses; half-life (t_{1/2}) was approximately 36-50 hours following the first dose; AUC increased generally dose proportional, Cₘₐₓ increased less than dose proportional; accumulation of the non-lactose dry powder formulation was moderate for Cₘₐₓ and AUC_{0-T} ranging from 1.95 to 2.27; and state was between days 7 and 14.

The initial high systemic exposure from administering the non-lactose dry powder indicates good lung delivery and dissolution. In contrast, the low systemic exposure on Day 1 with the lactose blend formulation suggests slow dissolution and a greater amount of lung binding. With repeat doses of the non-lactose dry powder formulation low accumulation was observed, indicating more consistent kinetics that would lower risks for adverse events with long term dosing. Steady state was achieved at about Day 7 when administering the non-lactose drug powder formulation, which would allow dose adjustment in a relatively short timeframe. On the other hand, there was higher exposure by Day 7 and Day 14 using the lactose blend formulation, suggesting significant lung accumulation driving systemic exposure. It is expected that steady state would not be achieved until about day 28 with the lactose blend formulation, based on modelling data.

### Data Modelling

Using clinical and non-clinical pharmacokinetic parameters obtained from studies with Formulation X and XI, and lactose blend formulations, it was possible to model lung exposure levels of Compound A in humans receiving either a non-lactose formulation of the present disclosure or a lactose blend.

Modelling of lung exposure utilized modified proprietary physiological-based pharmacokinetic modeling (PBPK) software (Gastro-Plus) to build a human kinetic model based primarily on rat nonclinical systemic and lung exposure data. The model is developed for the rat and then applied to the clinical exposure data. Parameters are adjusted as needed to ensure best possible fit to the measured systemic data in order to model the lung exposure and any addition projected exposure data based on modified doses or dose regimens.

This modelling indicates that the lung concentration following administration of Compound A as a lactose blend is between about 5- to 10-fold higher than that following a non-lactose formulation of the present disclosure (e.g., Formulation X or XI). Additionally, modelling reveals much more rapid clearance of the non-lactose formulation from the lungs, appearing to be substantively cleared after each dose, compared to the lactose blend which accumulates in the lung over time.

1. (canceled)

2. A respirable dry powder comprising respirable dry particles that comprise an active ingredient and one or more excipients, wherein
the active ingredient is Compound A; 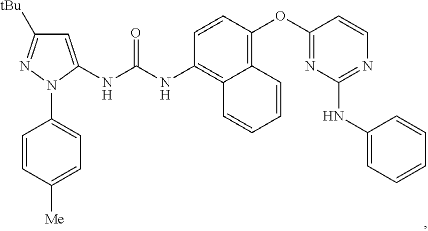 [SMILES: Cc1ccc(-n2nc(C(C)(C)C)cc2NC(=O)Nc2ccc(Oc3ccnc(Nc4ccccc4)n3)c3ccccc23)cc1]
or a pharmaceutically acceptable salt or tautomer thereof;
the active ingredient is present in an amount of about 0.5% to about 20% by weight; and
the one or more excipients comprise mannitol and sodium sulfate in an amount of about 4:1 to about 1:4 (wt %:wt %).

3. The dry powder of claim 2, further comprising a stabilizer, wherein the ratio of the stabilizer to the active ingredient is about 1:5 to about 1:20 (wt %:wt %).

4. The dry powder of claim 2, wherein Compound A is in a crystalline particulate form.

5. The dry powder of claim 4, wherein the crystalline particulate form is a nano-crystalline form.

6. (canceled)

7. The dry powder of claim 5, wherein the nano-crystalline form comprises a sub-particle with a Dv50 of about 50 nm to about 500 nm.

8-11. (canceled)

12. The dry powder of claim 2, wherein the active ingredient is present in an amount of about 2% to about 12% by weight.

13. (canceled)

14. The dry powder of claim 3, wherein the stabilizer is polysorbate 80.

15-16. (canceled)

17. The dry powder of claim 14, wherein the ratio of the stabilizer to the active ingredient is about 1:10 (wt %:wt %).

18. (canceled)

20. The dry powder of claim 14, wherein the polysorbate 80 is present in an amount of about 0.2% to less than 5% by weight.

21-28. (canceled)

29. A dry powder pharmaceutical formulation comprising homogenous respirable dry particles that comprise:
a) Compound A: 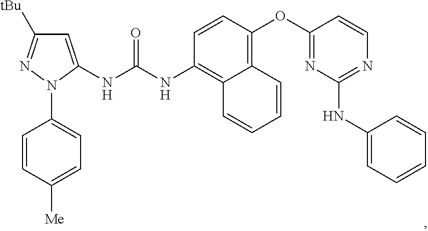 [SMILES: Cc1ccc(-n2nc(C(C)(C)C)cc2NC(=O)Nc2ccc(Oc3ccnc(Nc4ccccc4)n3)c3ccccc23)cc1]
or a pharmaceutically acceptable salt or tautomer thereof, in a nanocrystalline form (2%), polysorbate 80 (0.2%), mannitol (48.9%), and sodium sulfate (48.9%), all weight %;
b) Compound A: 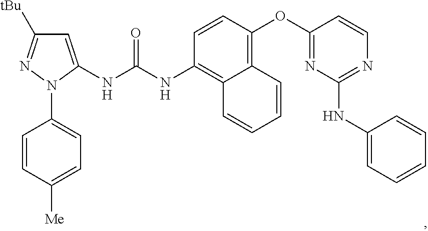 [SMILES: Cc1ccc(-n2nc(C(C)(C)C)cc2NC(=O)Nc2ccc(Oc3ccnc(Nc4ccccc4)n3)c3ccccc23)cc1]
or a pharmaceutically acceptable salt or tautomer thereof, in a nanocrystalline form (5%), polysorbate 80 (0.5%), mannitol (47.25%), and sodium sulfate (47.25%), all weight %;
c) Compound A: 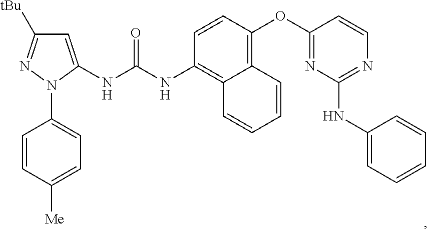 [SMILES: Cc1ccc(-n2nc(C(C)(C)C)cc2NC(=O)Nc2ccc(Oc3ccnc(Nc4ccccc4)n3)c3ccccc23)cc1]
or a pharmaceutically acceptable salt or tautomer thereof, in a nanocrystalline form (10%), polysorbate 80 (1.0%), mannitol (44.5%), and sodium sulfate (44.5%), all weight %;
d) Compound A: 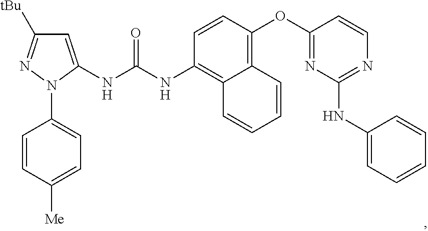 [SMILES: Cc1ccc(-n2nc(C(C)(C)C)cc2NC(=O)Nc2ccc(Oc3ccnc(Nc4ccccc4)n3)c3ccccc23)cc1]
or a pharmaceutically acceptable salt or tautomer thereof, in a nanocrystalline form (7.6%), polysorbate 80 (0.8%), mannitol (44.5%), and sodium sulfate (44.5%), all weight %; or
e) Compound A: 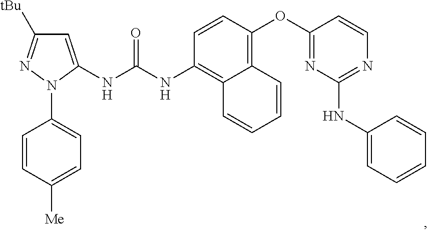 [SMILES: Cc1ccc(-n2nc(C(C)(C)C)cc2NC(=O)Nc2ccc(Oc3ccnc(Nc4ccccc4)n3)c3ccccc23)cc1]
or a pharmaceutically acceptable salt or tautomer thereof, in a nanocrystalline form (8.0%), polysorbate 80 (1.0%), mannitol (44.5%), and sodium sulfate (44.5%), all weight %.

30. The dry powder of claim 2, wherein the dry powder comprises homogenous respirable dry particles that comprise:
a) Compound A: 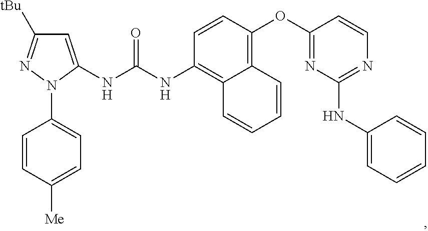 [SMILES: Cc1ccc(-n2nc(C(C)(C)C)cc2NC(=O)Nc2ccc(Oc3ccnc(Nc4ccccc4)n3)c3ccccc23)cc1]
or a pharmaceutically acceptable salt or tautomer thereof, in a nanocrystalline form (5%), polysorbate 80 (0.5%), mannitol (47.25%), and sodium sulfate (47.25%), all weight %; or
b) Compound A: 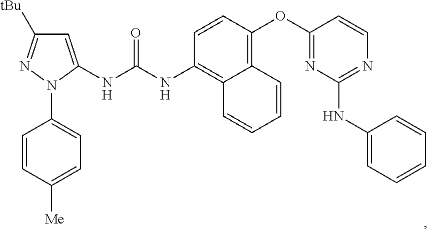 [SMILES: Cc1ccc(-n2nc(C(C)(C)C)cc2NC(=O)Nc2ccc(Oc3ccnc(Nc4ccccc4)n3)c3ccccc23)cc1]
or a pharmaceutically acceptable salt or tautomer thereof, in a nanocrystalline form (10%), polysorbate 80 (1.0%), mannitol (44.5%), and sodium sulfate (44.5%), all weight %.

31. (canceled)

32. The dry powder of claim 2, wherein the respirable dry particles have:
(a) a volume median geometric diameter (VMGD) of about 5 microns or less;
(b) a tap density of about 0.2 g/cc or greater;
(c) a 1 bar/4 bar dispersibility ratio (1/4 bar) of less than about 1.5 as measured by laser diffraction; or
(d) a 0.5 bar/4 bar dispersibility ratio (0.5/4 bar) of about 1.5 or less as measured by laser diffraction.

33-35. (canceled)

36. The dry powder of claim 2, wherein the dry powder has:
(a) mass median aerodynamic diameter (MMAD) of between about 1 micron and about 5 microns; or
(b) a fine particle fraction (FPF) of the total dose less than 5 microns of about 25% or more.

37-39. (canceled)

40. The dry powder of claim 2, wherein the dry powder is delivered to a subject with a capsule-based passive dry powder inhaler.

41. The dry powder of claim 2, wherein the dry powder or dry particles does not comprise lactose; or wherein the dry powder is not a lactose blend.

42. A method for treating or preventing an exacerbation of a chronic respiratory disease in a subject with a chronic respiratory disease, comprising administering to the subject an effective amount of a dry powder of claim 2.

43. A method for treating or preventing a condition selected from: COPD, asthma, pediatric asthma, cystic fibrosis, sarcoidosis, idiopathic pulmonary fibrosis, allergic rhinitis, rhinitis, sinusitis, allergic conjunctivitis, conjunctivitis, allergic dermatitis, contact dermatitis, psoriasis, ulcerative colitis, inflamed joints secondary to rheumatoid arthritis or osteoarthritis, rheumatoid arthritis, pancreatitis, cachexia, lung cancer, inhibition of the growth and metastasis of tumors including non-small cell lung carcinoma, breast carcinoma, gastric carcinoma, colorectal carcinomas and malignant melanoma, comprising administering to a subject in need thereof an effective amount of a dry powder of claim 2.

44. A method for treating or preventing a respiratory viral infection in a subject with a chronic condition or in an immunosuppressed subject, comprising administering to the subject an effective amount of a dry powder of claim 2.

45. A method for treating or preventing a condition selected from exacerbation of COPD and exacerbation of asthma, comprising administering to a subject in need thereof an effective amount of a dry powder of claim 2.

46. The method of claim 4E, wherein the exacerbation of COPD or exacerbation of asthma is a virally induced exacerbation.

47. A method for treating an exacerbation of inflammatory disease in a subject with a chronic condition selected from a group consisting of congestive heart failure, diabetes, cancer and a condition suffered by an immunosuppressed subject, comprising administering to the subject an effective amount of a dry powder of claim 2.

48. A method for treating or preventing an acute exacerbation of a respiratory disease, comprising administering to a subject in need thereof an effective amount of a dry powder of claim 2.

49-50. (canceled)

51. A method for treating or preventing exacerbations in a subject with a chronic respiratory disease, comprising administering to the subject an effective amount of a dry powder of claim 30.

52. A method for treating or preventing COPD, comprising administering to a subject in need thereof an effective amount of a dry powder of claim 30.
